(19) United States
(12) Patent Application Publication
(10) Pub. No.: US 2003/0211501 A1
(43) Pub. Date: Nov. 13, 2003

(54) Method and system for determining haplotypes from a collection of polymorphisms

(76) Inventors: J. Claiborne Stephens, Guilford, CT (US); Andreas Windemuth, Woodbridge, CT (US)

Correspondence Address: MORGAN & FINNEGAN, L.L.P., 345 PARK AVENUE, NEW YORK, NY, 10154, US

(21) Appl. No.: 10/258,155

(22) PCT Filed: Apr. 18, 2001
(86) PCT No.: PCT/US01/12831

(51) Int. Cl. G06F 17/60; C12Q 1/68; G06F 19/00; G01N 33/48; G01N 33/50

(52) U.S. Cl. 435/6; 702/20; 705/3

(57) ABSTRACT

Methods, computer programs and databases for determining haplotypes from a collection of polymorphisms are provided. These include methods, programs, and databases to find and measure the frequency of haplotypes in the general population; and methods, programs, and databases for predicting an individual's haplotypes from the individual's genotype for a gene.

### FIELD OF THE INVENTION

The invention relates to the field of genomics, and genetics, including genome analysis and the study of DNA variation. In particular, the invention relates to the field of predicting haplotype information from unphased and/or incomplete genotype information for an organism. The invention is particularly useful in the human health care, veterinary and agricultural fields.

### BACKGROUND OF THE INVENTION

The investigation of haplotypes began when it was recognized that certain pairs of loci violated Mendel's second law: rather than the independent segregation of variants at separate loci, there was a correlation in the transmission pattern from one locus to the next. Such correlated variants are called “haplotypes”. Haplotypes have historically had greatest importance in the analysis of pedigree data. More recently, with the capacity to generate DNA sequence information for a large number of individuals, “haplotype” has come to mean the specific sequence of alternative variants (e.g., single nucleotide polymorphisms or “SNPs”) at the polymorphic sites, often coming from a contiguous piece of DNA. In such applications attention has been diverted from family pedigrees to population samples, so there has been considerable interest in obtaining haplotypes when there is no recourse to familial transmission patterns. A number of molecular mechanisms have been described, such as sperm typing, single molecule dilution, cloning, or allele-specific amplification (AS-PCR), but all are currently limited to research investigations.

As early as 1971, it was realized that the ambiguity inherent in multilocus, but unphased, genotypes could be evaluated and at least partially overcome by statistical estimation of haplotype frequencies in a population. The first implementation of an algorithm for resolving phase of genotypes (Hill, 1975) is based on Hill's theory for two loci (Hill, 1974), each with two alleles, in which case an explicit maximum likelihood solution for haplotype frequencies exists. More recently, there have been extensions of that theory for additional loci and multiple alleles (Clark, 1990; Long et al., 1995; Excoffier and Slatkin, 1995; Hawley and Kidd, 1995). The focus of such algorithms is generally on the statistical estimation of population haplotype frequencies, and will be reviewed below.

Three algorithms published in 1995 (Excoffier and Slatkin, 1995; Hawley and Kidd, 1995; Long et al., 1995) all use the Expectation-Maximization (EM) algorithm for estimating haplotype frequencies in a population. The EM algorithm was originally proposed in 1977 (Dempster et al., 1977) as a general method of obtaining maximum likelihood estimates from data that are incomplete in some sense. In the application to haplotype frequency estimation, the incompleteness is the phase of the multiply heterozygous individuals. The paper by Long et al. goes beyond haplotype frequency estimation to construct a model framework for testing the statistical association among loci. Furthermore, it makes allowance for the possibility of null alleles at one or more loci. The model and algorithm are described for three loci, although they claim applicability to more complicated situations. The paper by Hawley and Kidd deals explicitly with multiple populations, but again this reference is focused primarily on frequency estimation. The mathematical basis of a maximum likelihood approach to haplotype estimation is explained well in the paper by Excoffier and Slatkin (1995). Although the latter paper mentions, as a potential application, “inferring which gametes are most likely associated to form genotypes in all sampled individuals”, it does not say how this can be done. All three methods based on the EM algorithm require multiple starting conditions to facilitate finding the true maximum-likelihood solution, and there is still no guarantee that the true maximum will be found. These published algorithms also have the very pragmatic problem of being limited by at least one of the following: the maximum number of polymorphic positions, possible haplotypes, or heterozygous sites in an individual.

The inventors herein are aware of only one reference (Clark 1990) that discloses an algorithm for assigning haplotypes to unrelated individuals in a population sample. It proceeds by assigning haplotypes that are observed as homozygotes or single-site heterozygotes, then interrogating whether one (or more) of these is consistent with an ambiguous individual, that is, an individual heterozygous at two or more sites. It is an order-dependent algorithm, in that different orders give different answers, and so must be applied several times to look for differences and a single best answer. This reference has identified three main problems in applying the algorithm: 1) it might never get started, if there are no unambiguous individuals; 2) it might not be able to resolve every individual in a sample; and 3) it might resolve certain individuals incorrectly. Indeed, the reference included the results of computer simulations that evaluate the severity of these potential problems. Also, in a recent application of Clark's algorithm to the LPL locus (Clark et al. 1998), the algorithm required supplementation by AS-PCR, a molecular technique for resolving haplotypes, since every individual in the sample was a heterozygote.

None of the prior art disclosed or suggested an approach for assigning haplotypes to unrelated individuals that was amenable to a high-throughput mode of analysis. Moreover, none of the prior art disclosed or suggested an approach for incorporating error analysis or for estimating missing data. Finally, none of the prior art disclosed or suggested a process that would not require multiple starting conditions, nor did they disclose or suggest a process that would be amenable to the complications implicit in data with dozens of polymorphic loci. Thus, there is a need to develop a process that assigns haplotype pairs to unrelated individuals; prioritizes automation, robustness, and statistical evaluation of the accuracy of the results; and has the capacity to cope with data of substantially greater complexity than that addressed in prior art.

The methods and tools described herein provide processes for predicting haplotypes and haplotype pairs from unphased and/or incomplete genotype data. The processes are preferably carried out with the aid of a computer.

The exemplified methods and tools are partially embodied in a computer program coupled to a database used to display and analyze haplotype, genotype and related statistical information. It includes novel graphical and computational methods for treating haplotypes, genotypes, and related data in a consistent and easy-to-interpret manner.

### SUMMARY OF THE INVENTION

The invention relates to a process for deriving the presence and frequency of haplotypes from a collection of genotypes of several individual polymorphisms in a locus, measured for a sample group of individuals. The process begins with an exhaustive enumeration (expansion) of all possible haplotypes (called the “Hap Expansion” phase), then proceeds through a self-consistent, iterative process to deduce the haplotypes most likely to be present, and the most likely assignment of haplotype pairs to each individual (called the “Hap Assignment” phase). The process also results in a probability score specifying the likelihood of the result being correct. The process takes advantage of family relationships among the sample group, but does not require them. The process is embodied in the HAP™ Builder program, a computer code written in Java providing an interface for a skilled person to efficiently carry out the process and store the results.

More specifically, the invention relates to a method and tools for assigning haplotype pairs for a polymorphic genomic region to a plurality of individuals, comprising:

(a) obtaining a genotype for the polymorphic genomic region from each of the individuals;

(b) enumerating all possible haplotypes hᵢ that are consistent with each genotype;

(c) assigning an evidence score sᵢ to each of the enumerated haplotypes hᵢ;

(d) calculating an initial haplotype frequency fᵢ for each haplotype among the possible haplotypes, wherein the initial haplotype frequency fᵢ is a function of the evidence score sᵢ;

(e) determining for each genotype obtained in step (a) a pair score Fₖ for each pair of haplotypes that is consistent with that genotype, wherein Fₖ is a function of the frequency fᵢ for each of the haplotypes in the pair;

(f) calculating, for each genotype and consistent haplotype pair whose pair score Fₖ meets a pair score criterion, a probability pₖ that assignment of that haplotype pair to the genotype would be correct;

(g) generating a revised haplotype frequency fᵢ for each haplotype, wherein the revised haplotype frequency fᵢ is a function of the probability pₖ for each consistent haplotype pair which contains the haplotype; and

(h) repeating steps (e) through (g) until an end condition is reached, with the proviso that for each repetition the frequency fᵢ employed in step (e) is replaced by the revised frequency fᵢ determined in step (g).

Steps (a) though (d) are called the initiation, or Hap Expansion, phase of the method. Steps (a), (b) and (c) can be performed for one individual at a time or in parallel.

Steps (e) through (g) are called the Hap Assignment phase of the method. Steps (e) and (f) can be performed for one genotype at a time or in parallel.

The invention also relates to a method and tools for assigning a haplotype pair to a polymorphic genomic region of an individual, comprising:

(a) obtaining the genotype for the polymorphic genomic region from the individual;

(b) enumerating all possible haplotypes hᵢ for the genotype;

(c) providing a frequency fᵢ for each of the possible haplotypes, where fᵢ is determined by the method for assigning haplotype pairs for a polymorphic genomic region to a plurality of individuals, as discussed herein;

(d) determining a pair score Fₖ for each pair of possible haplotypes hᵢ that are consistent with the genotype, wherein Fₖ is a function of the frequency fᵢ for each of the haplotypes in the pair; and

(e) assigning to the genotype the haplotype pair having the highest pair score Fₖ.

The invention also relates to methods and tools for predicting the probable haplotypes and haplotype pairs of one or more loci of an individual. The invention also relates to methods and tools for estimating the probability that the predicted haplotypes and haplotype pairs are correct.

The invention also relates to a method and tools for filling in missing genotype data for any individual and polymorphic site that was not or could not be measured, comprising using the most probable assignment of haplotypes determined by the methods of the invention to construct the most likely genotype for the individual.

The invention also relates to methods of constructing a haplotype database for a population containing reference haplotype pair frequency data. The invention also relates to methods of predicting the presence of a haplotype pair in an individual using such a database. The methods comprise accessing the database containing reference haplotype pair frequency data to determine a probability, for each of the possible haplotype pairs, that the individual has the possible haplotype pair; and analyzing the determined probabilities to predict haplotype pairs for the individual.

The methods and tools of the invention make it possible to determine haplotypes and haplotype pairs in an individual, or in a plurality of individuals, based on unphased and/or incomplete genotype information. The individuals may be part of a population such as the general population, an ethno-geographic group, or a clinical or disease population, or they may all be of the same gender.

Similarly, in agricultural biotechnology, the method and tools of the invention can be used to determine the haplotypes and haplotype pairs of genes responsible for specific desirable traits, e.g., drought tolerance and/or improved crop yields, and reduce the time and effort needed to transfer desirable traits.

The invention includes methods, computer programs and databases to analyze and make use of genotype information to deduce and/or predict haplotype information. These include methods, programs, and databases for finding and measuring the frequency of haplotypes and/or haplotype pairs in a population; and methods, programs, and databases for inferring an individual's haplotype from the individual's genotype.

### BRIEF DESCRIPTION OF THE DRAWINGS

FIGS. 1A and 1B. System Architecture Schematic.

FIG. 2. First part of Flow Chart for a method and system for determining haplotypes from a collection of polymorphisms.

FIG. 3. Second part of Flow Chart for a method and system for determining haplotypes from a collection of polymorphisms.

FIG. 4. Third part of Flow Chart for a method and system for determining haplotypes from a collection of polymorphisms.

FIG. 5. DecoGen HAP™ Builder View. The top half of FIG. 5 is a screen showing a set of candidate genes for which polymorphism data has been obtained or is in the process of being obtained, and which may be selected for being haplotyped. The columns on the right side of the screen indicate various stages in the process of analyzing target regions of the gene identified in the corresponding row. The various colors provide an immediate visual indicator of the status of the gene at each stage of analysis. The bottom part of this figure is a screen which provides information concerning the sequencing of various regions of the selected candidate gene.

FIG. 6. Gene Structure View. This screen shows the location of features in the gene (such as promoter, introns, exons, etc.), as well as actual sequence data, for a gene for which the “Anno” column has been selected in the screen of FIG. 5.

FIG. 7. Gene Haplotypes View. The screen in the top right side of this view shows information about the polymorphic sites in a gene for which the “Haplo” column has been selected in the screen of FIG. 5, such as the location of the polymorphic sites, the type of polymorphism, and an indication of the frequency with which each polymorphism has been seen in various world population groups. The screen includes boxes which may be checked to include the polymorphic site in a haplotype analysis.

FIG. 8. Gene Haplotypes View (Cont.). The screen in the top left side of this view shows an items selection menu which results after the “Edit” item is clicked on in FIG. 7. The “DeHarv” menu item is highlighted.

FIG. 9. Gene Haplotypes View (Cont.). The screen in the top right side of this view shows information which results after the “DeHARV” menu item is selected in the “Edit” menu on the top left side of FIG. 8; only six of the polymorphic sites shown in the screen in the top right of FIG. 8 are selected in the screen in the top right side of FIG. 9.

FIG. 10. Gene Haplotypes View (Cont.). This view shows screens which result when the “Filter Polymorphisms” menu item is selected in the Edit menu of FIG. 9 (which is not shown open in FIG. 9). The box in the middle right side of this view labeled “ScoredDiplotype Objects” shows the unphased genotypes of subjects in the database and their ethno-geographic origin. The screen in the bottom right side of this view labeled “ScoredHaplotypes Objects” shows the expanded haplotypes enumerated from the genotypes in the middle screen for each of the selected (accepted) polymorphic sites.

FIG. 11. Gene Haplotypes View (Cont.). This view shows screens which result when the “Assign” menu item in the edit menu of FIG. 10 (which is not shown open in FIG. 10) is selected one time. The screen in the middle of the figure labeled “Scored Diplotype Objects” shows the “Hap1” and “Hap2” pair assignments (i.e., the genotype to haplotype resolution) for each of the individuals in the population being examined after several iterations of the HAP™ Builder algorithm, as well as the HapPair Score assigned to them. The window labeled “Scored Haplotype Objects” (shown in the lower right side of the view in FIG. 11) provides the different haplotypes determined in the examined population, with a haplotype frequency score, as well as the number of times each haplotype is seen in the entire population and in the various population groups.

FIG. 12. Gene Haplotypes View (Cont.). This view shows a window labeled “HapPair Objects” which is displayed as a result of clicking on the “Score” cell for row 94 (individual UP002) in the “ScoredDiplotypes Objects” box in the center of FIG. 12. This window contains the 15 most likely haplotype pairs for subject UP002 based on the current haplotype pair scores.

FIG. 13. Gene Haplotypes View (Cont.). This view shows screens which result after the “Assign” command in the Edit menu in FIG. 12 has been invoked multiple times.

FIG. 14. Gene Haplotypes View (Cont.). This view shows a screen with “warnings” (e.g., missing genotype data) highlighted in light gray. This view also shows a screen with the icon for the individual UP002 highlighted in dark gray in the family tree schematic because the Mendelian inheritance rules are violated.

FIG. 15. Gene Haplotypes View (Cont.). This view shows a window labeled “15 HapPair Objects” which results when subject UP002 is selected in the Scored DiplotypeObjects list.

### DETAILED DESCRIPTION OF THE INVENTION

I. DEFINITIONS

In the context of this disclosure, the following terms shall be defined as follows unless otherwise indicated:

Allele—A particular form of a genetic locus, distinguished from other forms by its particular nucleotide sequence.

Ambiguous polymorphic site—A heterozygous polymorphic site or a polymorphic site for which nucleotide sequence information is lacking.

Candidate Gene—A gene which is hypothesized to be responsible for a disease, condition, or the response to a treatment, or to be correlated with one of these.

Gene—A segment of DNA that contains all the information for the regulated biosynthesis of an RNA product, including promoters, exons, introns, and other untranslated regions that control expression.

Genotype—An unphased 5′ to 3′ sequence of nucleotide pair(s) found at one or more polymorphic sites in a locus on a pair of homologous chromosomes in an individual. As used herein, genotype includes a fall-genotype and/or a sub-genotype as described below.

Full-genotype—The unphased 5′ to 3′ sequence of nucleotide pairs found at all known polymorphic sites in a locus on a pair of homologous chromosomes in a single individual.

Sub-genotype—The unphased 5′ to 3′ sequence of nucleotides seen at a subset of the known polymorphic sites in a locus on a pair of homologous chromosomes in a single individual.

Genotyping—A process for determining a genotype of an individual.

Haplotype—A 5′ to 3′ sequence of nucleotides found at one or more polymorphic sites in a locus on a single chromosome from a single individual. As used herein, haplotype includes a fall-haplotype and/or a sub-haplotype as described below.

Full-haplotype—The 5′ to 3′ sequence of nucleotides found at all known polymorphic sites in a locus on a single chromosome from a single individual.

Sub-haplotype—The 5′ to 3′ sequence of nucleotides seen at a subset of the known polymorphic sites in a locus on a single chromosome from a single individual.

Haplotype pair—The two haplotypes found for a locus in a single individual.

Haplotyping—A process for determining one or more haplotypes in an individual and includes use of family pedigrees, molecular techniques and/or statistical inference.

Haplotype data—Information concerning one or more of the following for a specific gene: a listing of the haplotype pairs in each individual in a population; a listing of the different haplotypes in a population; frequency of each haplotype in that or other populations, and any known associations between one or more haplotypes and a trait.

Isoform—A particular form of a gene, mRNA, cDNA or the protein encoded thereby, distinguished from other forms by its particular sequence and/or structure.

Isogene—One of the isoforms of a gene found in a population. An isogene contains all of the polymorphisms present in the particular isoform of the gene.

Isolated—As applied to a biological molecule such as RNA, DNA, oligonucleotide, or protein, isolated means the molecule is substantially free of other biological molecules such as nucleic acids, proteins, lipids, carbohydrates, or other material such as cellular debris and growth media. Generally, the term “isolated” is not intended to refer to a complete absence of such material or to absence of water, buffers, or salts, unless they are present in amounts that substantially interfere with the methods of the present invention.

Locus—A location on a chromosome or DNA molecule corresponding to a gene or a physical or phenotypic feature.

Naturally-occurring—A term used to designate that the object it is applied to, e.g., naturally-occuring polynucleotide or polypeptide, can be isolated from a source in nature and which has not been intentionally modified by man.

Nucleotide pair—The nucleotides found at a polymorphic site on the two copies of a chromosome from an individual.

Phased—As applied to a sequence of nucleotide pairs for two or more polymorphic sites in a locus, phased means the combination of nucleotides present at those polymorphic sites on a single copy of the locus is known.

Polymorphic genomic region—A region comprising one or more polymorphic sites in a single contiguous region or in two or more noncontiguous regions of a single chromosome.

Polymorphic site (PS)—A position within a locus at which at least two alternative sequences are found in a population, the most frequent of which has a frequency of no more than 99%.

Polymorphic variant—A gene, mRNA cDNA, polypeptide or peptide whose nucleotide or amino acid sequence varies from a reference sequence due to the presence of a polymorphism in the gene.

Polymorphism—The sequence variation observed in an individual at a polymorphic site. Polymorphisms include nucleotide substitutions, insertions, deletions and microsatellites and may, but need not, result in detectable differences in gene expression or protein function.

Polymorphism data—Information concerning one or more of the following for a specific gene: location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene.

Polymorphism Database—A collection of polymorphism data arranged in a systematic or methodical way and capable of being individually accessed by electronic or other means.

Polynucleotide—A nucleic acid molecule comprised of single-stranded RNA or DNA or comprised of complementary, double-stranded DNA.

Population Group—A group of individuals sharing a common ethnogeographic origin.

Reference Population—A group of subjects or individuals who are predicted to be representative of the genetic variation found in the general population. Typically, the reference population represents the genetic variation in the population at a certainty level of at least 85%, preferably at least 90%, more preferably at least 95% and even more preferably at least 99%.

Single Nucleotide Polymorphism (SNP)—Typically, the specific pair of nucleotides observed at a single polymorphic site. In rare cases, three or four nucleotides may be found.

Subject—An individual whose genotypes or haplotypes or response to treatment or disease state are to be determined.

Treatment—A stimulus administered internally or externally to a subject.

Unphased—As applied to a sequence of nucleotide pairs for two or more polymorphic sites in a locus, unphased means the combination of nucleotides present at those polymorphic sites on a single copy of the locus (i.e., located on a single DNA strand) is not known.

II. METHODS OF IMPLEMENTING THE INVENTION

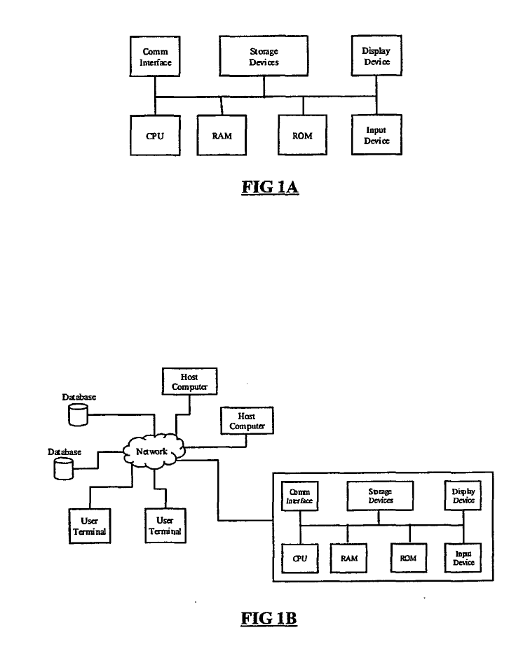

The present invention may be implemented with a computer, an example of which is shown in FIG. 1A. The computer includes a central processing unit (CPU) connected by a system bus or other connecting means to a communication interface, system memory (RAM), non-volatile memory (ROM), and one or more other storage devices such as a hard disk drive, a diskette drive, and a CD ROM drive. The computer may also include an internal or external modem (not shown). The computer also includes a display device, such as a CRT monitor or an LCD display, and an input device, such as a keyboard, mouse, pen, touch-screen, or voice activation system. The computer stores and executes various programs such as an operating system and application programs. The computer may be embodied, for example, as a personal computer, work station, laptop, mainframe, or a personal digital assistant. The computer may also be embodied as a distributed multi-processor system or as a networked system such as a LAN having a server and client terminals.

The present invention uses a program, referred to as the HAP™ Builder program, that generates views (or screens) displayed on a display device and which the user can interact with to accomplish a variety of tasks and analyses. For example, the HAP™ Builder program allows users to view and analyze large amounts of information such as subject identifiers (e.g., subject number or cell line number); gene-related data (e.g., gene name, gene symbol, GenBank accession number); family data (e.g., family number, father, mother, number of siblings); polymorphism data (e.g., region, position, nucleotide changes (i.e., the polymorphic nucleotide(s) as compared to the reference nucleotide(s)); genotype data (e.g., scored diplotype objects); haplotype data (e.g. haplotype identifiers, haplotype frequencies, haplotype pairs, and haplotype pair scores (indicating the probability that the haplotype pair of an individual is correct)); and population data (e.g., ethnic, geographical, clinical, and genotype and haplotype data for various populations).

The HAP™ Builder program is preferably written in the Java programming language. However, the program may be written using any conventional programming language such as for example C, C++, Visual Basic™ or Visual Pascal™. The HAP™ Builder program may be stored and executed on the computer. It may also be stored and executed in a distributed manner.

The data processed by the HAP™ Builder program is preferably stored as part of a relational database (e.g., an instance of an Oracle™ database or a set of ASCII flat files). This data can be stored on, for example, a CD ROM or in one or more storage devices accessible by the computer. The data may be stored on one or more databases in communication with the computer via a network.

In one scenario, the data will be delivered to the user on any standard media (e.g., CD, floppy disk, tape) or can be downloaded over the internet. The HAP™ Builder program and data may also be installed on a local machine. The HAP™ Builder program and data will then be on the machine that the user directly accesses.

FIG. 1B shows an implementation where a network interconnects one or more host computers with one or more user terminals. The communication network may, for example, include one or more local area networks (LANs), metropolitan area networks (MANs), wide area networks (WANs), or a collection of interconnected networks such as the Internet. The network may be wired, wireless, or some combination thereof. The host computer may, for example, be a world wide web server (“web server”). The user terminal may, for example, be a client device such as the computer shown in FIG. 1A.

A web server stores information documents called pages. A server process listens for incoming connections from clients (e.g., browsers running on a client device). When a connection is established, the client sends a request and the server sends a reply. The request typically identifies a page by its Uniform Resource Locator (URL) and the reply includes the requested page. This client-server protocol is typically performed using the hypertext transfer protocol (“http”). Pages are viewed using a browser program. They are written in a language called hypertext markup language (“html”). A typical page includes text and formatting comments called tags. Pages may also include links (pointers) to other pages. Strings of text or images that are links to other pages are called hyperlinks. Hyperlinks are highlighted (e.g., by color, underlining) and may be invoked by placing the cursor on the highlighted area and selecting it (e.g., by clicking the mouse button). A page may also contain a URL reference to a portion of multimedia data such as an image, video segment, or audio file. Pages may also point to a Java program called an applet. When the browser connects to where the applet is stored, the applet is downloaded to the client device and executed there in a secure manner. Pages may also contain forms that prompt a user to enter information or that have active maps. Data entered by a user may be handled by common gateway interface (CGI) programs. Such programs may, for example, provide web users with access to one or more databases.

As shown in FIG. 1B the host computer may include a CPU connected by a system bus or other connecting means to a communication interface, system memory (RAM), nonvolatile memory (ROM), and a mass storage device. The mass storage device may, for example, be a collection of magnetic disk drives in a RAID system. The mass storage device may, for example, store the aforementioned web pages, applets, and the like. The host computer may also include an input device, such as a keyboard, and a display device to allow for control and management by an administrator. Additionally, the host computer may be connected to additional devices such as printers, auxiliary monitors or other input/output devices. The input device and display device may also be provided on another computer coupled to the host computer. The host computer may be embodied, for example, as one or more mainframes, workstations, personal computers, or other specialized hardware platforms. The functionality of the host computer may be centralized or may be implemented as a distributed system. As also shown in FIG. 1B, the host computer may communicate with one or more databases stored on any of a variety of hardware platforms.

In an Internet embodiment, for example involving the system of FIG. 1B, the HAP™ Builder program will be web-based and will be delivered as an applet that runs in a web browser. In this case, the data will reside on a server machine and will be delivered to the HAP™ Builder program using a standard protocol (e.g., HTTP with cgi-bin). To provide extra security, the network connection could use a dedicated line. Furthermore, the network connection could use a secure protocol such as Secure Socket Layer (SSL) which only provides access to the server from a specified set of IP addresses.

In another embodiment, the HAP™ Builder program can be installed on a user machine and the data can reside on a separate server machine. Communication between the two machines can be handled using standard client-server technology. An example would be to use TCP/IP protocol to communicate between the client and an oracle server.

It may be noted that in any of the prior scenarios, some or all of the data used by the HAP™ Builder program could be directly imported into the HAP™ Builder program by the user. This import could be carried out by reading files residing on the user's local machine, or by cutting and pasting from a user document into the interface of the HAP™ Builder program.

In yet a further embodiment, some or all of the data or the results of analyses of the data could be exported from the HAP™ Builder program to the user's local computer. This export could be carried out by saving a file to the local disk or by cutting and pasting to a user document.

In the present invention various calculations are performed to generate items displayed on a screen or to control items displayed on a screen. As is well known, some basic calculations may be performed using database query language (SQL), while other computations are performed by the HAP™ Builder program (i.e., the Java program which, as previously mentioned, may be an applet downloaded over the internet.)

III. METHODS OF THE INVENTION

The invention relates to a process for deriving the presence and frequency of haplotypes from a collection of genotypes of several individual polymorphisms in a gene locus, measured for a sample group of individuals. The process begins with an exhaustive enumeration (expansion) of all possible haplotypes (called the “Hap Expansion” phase), then proceeds through a self-consistent, iterative process to produce the haplotypes most likely to be present, as well as the most likely assignment of haplotype pairs to each individual (called the “Hap Assignment” phase). The process also results in a probability score specifying the likelihood of the result being correct. The process takes advantage of family relationships among the sample group, but does not require them. The process is embodied in the HAP™ Builder program, a computer code written in Java providing an interface for a skilled person to efficiently carry out the process and store the results.

More specifically, the invention relates to a method for assigning haplotype pairs for a polymorphic genomic region to a plurality of individuals, comprising:

(a) obtaining a genotype for the polymorphic genomic region from each of the individuals;

(b) enumerating all possible haplotypes hᵢ consistent with each genotype;

(c) assigning an evidence score sᵢ to each of the enumerated haplotypes hᵢ;

(d) calculating an initial haplotype frequency fᵢ for each haplotype among the possible haplotypes, wherein the initial haplotype frequency fᵢ is a function of the evidence score sᵢ;

(e) determining for each genotype obtained in step (a) a pair score Fₖ for each pair of haplotypes that are consistent with that genotype, wherein Fₖ is a function of the frequency fᵢ for each of the haplotypes in the pair;

(f) calculating, for each genotype and consistent haplotype pair whose pair score Fₖ meets a pair score criterion, a probability pₖ that assignment of that haplotype pair to the genotype would be correct;

(g) generating a revised haplotype frequency fᵢ for each of the haplotypes, wherein the revised haplotype frequency fᵢ is a function of the probability pₖ for each consistent haplotype pair which contains the haplotype; and

(h) repeating steps (e) through (g) until an end condition is reached, with the proviso that for each repetition the frequency fᵢ employed in step (e) is replaced by the revised frequency fᵢ determined in step (g).

Steps (a) though (d) are called the initiation, or Hap Expansion, phase of the method. Steps (a), (b) and (c) can be performed for one individual at a time or in parallel.

Steps (e) through (g) are called the Hap Assignment phase of the method. Steps (e) and (f) can be performed for one genotype at a time or in parallel.

In order to more efficiently use computing resources, particularly when large numbers of individuals are being haplotyped, it is preferred that the above procedure be modified as follows: After the genotypes are obtained (or, as they are obtained), they are combined into groups, where all the genotypes in each group are identical. Groups may optionally be characterized by one or more additional criteria, such as, for example, a requirement that all individuals from whom the genotypes are derived must belong to a single population group. Additional criteria may be, for example, a requirement that all individuals from whom the genotypes are derived must be of the same gender, or belong to a single clinical or disease population, or to a population exhibiting a particular response to a drug or other stimulus, or to a population characterized by a particular genotype or haplotype at some other polymorphic region. Thus, in this embodiment, all members of a group minimally have the same genotype, but there may be more than one group with the same genotype. The number of individuals sharing a distinct genotype within a group g is called the multiplier, n_{g}. Hap expansion is preferably carried out only once for each distinct (different) genotype, and the multiplier is used at the end of the expansion to give the appropriate weight to the frequency scores. In this preferred embodiment, the method comprises the steps of:

(a) obtaining a genotype for the polymorphic genomic region from each of the individuals;

(b) grouping the genotypes obtained in step (a) into groups, wherein in each group g there are n_{g} identical genotypes (any unique genotypes are regarded as groups having n_{g}=1);

(c) enumerating all possible haplotypes hᵢ that are consistent with each distinct genotype;

(d) assigning an evidence score sᵢ to each of the enumerated possible haplotypes hᵢ;

(e) for each group g, calculating an initial haplotype frequency (fᵢ) for each haplotype among the possible haplotypes, wherein the initial haplotype frequency fᵢ is a function of the product (sᵢ)(n_{g});

(f) determining, for each group g, a pair score Fₖ for each pair of haplotypes that is consistent with the genotype of that group, wherein Fₖ is a function of the frequency fᵢ for each of the haplotypes in the pair;

(g) calculating, for each genotype and consistent haplotype pair whose pair score Fₖ meets a pair score criterion, a probability pₖ that assignment of that haplotype pair to the genotype would be correct;

(h) generating a revised haplotype frequency fᵢ for each haplotype, wherein the revised haplotype frequency fᵢ is a function of the product (n_{g})(pₖ) for each consistent haplotype pair which contains the haplotype; and

(i) repeating steps (f) through (h) until an end condition is reached, with the proviso that for each repetition the frequency fᵢ employed in step (f) is replaced by the revised frequency fᵢ determined in step (h).

In this embodiment, steps (a) though (e) are the initiation, or Hap Expansion, phase of the method. Steps (a), (b) (c) and (d) can be performed for one individual (or group) at a time or in parallel. Steps (f) through (h) are the Hap Assignment phase of the method. Steps (f) and (g) can be performed for one group at a time or in parallel. Characterizing groups by one or more additional criteria may be done before or after the enumerating step, but is preferably done before the enumerating step.

The invention also relates to a method for predicting an individual's haplotype pair for a polymorphic genomic region, comprising:

(a) obtaining the genotype for the polymorphic genomic region from the individual;

(b) enumerating all possible haplotypes hᵢ for the genotype;

(c) providing a frequency fᵢ for each of the possible haplotypes, where fᵢ is determined by one of the methods described herein;

(d) determining a pair score Fₖ for each pair of possible haplotypes hᵢ that are consistent with the genotype, wherein Fₖ is a function mention of the frequency fᵢ for each of the haplotypes in the pair; and

(e) assigning to the genotype the haplotype pair having the highest pair score Fₖ.

The invention also relates to a method for assigning a haplotype pair for a polymorphic genomic region of an individual, comprising:

(a) obtaining the genotype for the polymorphic genomic region from the individual;

(b) enumerating all possible haplotypes hᵢ for the genotype;

(c) providing a frequency fᵢ for each of the possible haplotypes, where fᵢ has been previously determined by the method for assigning haplotype pairs for a polymorphic genomic region to a plurality of individuals discussed herein;

(d) determining a pair score Fₖ for each pair of possible haplotypes hᵢ that are consistent with the genotype, wherein Fₖ is a function of the frequency fᵢ for each of the haplotypes in the pair; and

(e) assigning to the genotype the haplotype pair having the highest pair score Fₖ.

In a preferred embodiment, the invention also relates to a method and tools for estimating the probability that the haplotype pairs assigned by the methods described immediately above are correct, comprising (a) the steps described immediately above, and further comprising (b) determining the probability score pₖ from the formula

$${p}_{k} = \frac{{F}_{k}}{{\sum }_{l = 0}^{{N}_{rank}} {F}_{l}} .$$

In the formula above, Nᵣₐₙₖ is the number of pairs of haplotypes selected by the practitioner for consideration, which is preferably a subset of all the possible consistent pairs. Typically, one would select only the Nᵣₐₙₖ highest scoring pairs for consideration.

The invention also relates to a method for filling in missing genotype data, comprising using, in the above-mentioned methods of the invention, the following genotype probabilities for any genotypes that could not be measured:

p_{c}=0.25, where p_{c} represents the probability that the genotype is homozygous for the more common allele,

pₕ=0.5, where pₕ represents the probability that the genotype is heterozygous, and

pᵣ=0.25, where pᵣ represents the probability that the genotype is homozygous for the less common allele;

and for any individual polymorphic site that could not be measured, using the most probable assignment of haplotypes resulting from the use of the probabilities p_{c}, pₕ, and pᵣ to construct the most likely genotype by combining the two haplotype alleles at this position.

The invention also relates to methods of constructing a haplotype database for a population, comprising:

(a) identifying individuals to include in the population;

(b) determining haplotype data for each individual in the population from genotype information;

(c) organizing the haplotype data for the individuals in the population into fields; and

(d) storing the haplotype data for individuals in the population according to the fields.

The invention also relates to methods of predicting the presence of a haplotype pair in an individual comprising, in order:

(a) obtaining a genotype for the individual;

(b) enumerating all possible haplotype pairs which are consistent with the genotype;

(c) accessing a database containing reference haplotype pair frequency data to determine a probability, for each of the possible haplotype pairs, that the individual has a possible haplotype pair; and

(d) analyzing the determined probabilities to predict haplotype pairs for the individual.

The methods and tools of the invention make it possible to determine haplotypes and haplotype pairs in an individual, or a plurality of individuals in a population, based on unphased and/or incomplete genotype information. The individuals may for example be of the same gender, and/or part of the general population, an ethno-geographic population group, a clinical or disease population, or a population exhibiting a particular response to a stimulus (e.g. a response to a drug). Frequency and probability scores for haplotypes and haplotype pairs are preferably calculated and used within the same population, but may be used across different populations and population groups if desired.

Similarly, in agricultural biotechnology, the method and tools of the invention can be used to determine the haplotypes and haplotype pairs of genes responsible for specific desirable traits, e.g., drought tolerance and/or improved crop yields, and reduce the time and effort needed to transfer desirable traits.

The invention includes methods, computer programs, and databases for analyzing and making use of genotype information to deduce and/or predict haplotype information. These include methods, programs, and databases for finding and measuring the frequency of haplotypes and/or haplotype pairs in a population; and methods, programs, and databases for predicting an individual's haplotypes from the individual's genotype.

Various aspects of the invention are discussed in further detail below.

A. POPULATION SIZE

In the methods of the invention relating to deriving the presence and frequency of haplotypes from a collection of genotypes, it is preferred that the minimum number of individuals being haplotyped be greater than the number of haplotypes expected from the number of polymorphisms in the loci being haplotyped. Based on an analysis of over 3500 genes, the present inventors have empirically determined that the number of haplotypes for a gene,, on average, is about 1.1 to 1.3 times the number of individual polymorphisms in the gene being studied (data not shown). For example, in a locus containing 15 polymorphisms, it is expected that the number of haplotypes in the general population is between about 17 and about 20 (i.e., 1.1×15=16.5 and 1.3×15=19.5); thus the number of individuals in a reference population being haplotyped should preferably be at least 20.

If on the other hand, the skilled artisan is interested in detecting all haplotypes for the polymorphic locus that exist in the general population above a fairly low frequency, then the size of the reference population should be sufficient to predict the existence of multiple copies of such haplotypes with high certainty. For example, in a sample of 100 individuals, a haplotype present in a frequency of 10% would be expected to occur in 19 individuals, once as a homozygote and 18 times as a heterozygote. Thus, for pharmacogenetic applications, it is desirable to use genotypes from about 100 unrelated individuals in the HAP™ Builder process described herein to establish the haplotypes that exist in the general population for a particular polymorphic locus of interest, e.g., a typical gene of pharmaceutical relevance. However, if establishing haplotypes for a very polymorphic locus, e.g., one that has >60 polymorphic sites, then it would be preferable to use a larger reference population, such as 200, 400, 600, 800, or up to about 1000 individuals.

B. HAP EXPANSION

Any given genotype may be heterozygous at any of the variable sites. If a genotype is found homozygous at all sites (e.g.: A/A, C/C, C/C, T/T), in the absence of genotyping error the only possible assignment is two simultaneous copies of the same haplotype (ACCT). If a genotype is heterozygous at one position (e.g.: A/A, C/C, C/G, T/T), there is likewise only one possible assignment, i.e. the combination of two haplotypes (ACCT and ACGT). If the genotype is heterozygous at more than one position, there are multiple assignments possible. The Hap Expansion constitutes a way of enumerating all possibly observed haplotypes and assigning to them a score that amounts to an initial estimate of their frequency.

Hap Expansion goes through the following steps:

(1) For each genotype, all possible combinations of haplotypes that are consistent with the genotype are determined. For a fully homozygous genotype, there will be one haplotype. For a singly heterozygous genotype there will be two. For a doubly heterozygous sample there will be four, etc. In general, if there are n heterozygous positions, there will be 2ⁿ haplotypes that are consistent with the observed genotype.

(2) Evidence scores are assigned to the haplotypes found in step (1). In an embodiment of the invention which is exemplified herein, each haplotype in the expansion will have an evidence score of 2/2ⁿ assigned to it. Thus, a homozygous genotype will generate one haplotype with score 2, a singly heterozygous genotype will generate two haplotypes with a score of 1 each, a doubly homozygous genotype will generate four haplotypes with a score of 0.5, etc. In this embodiment, 2ⁿ haplotypes with a score of 2/2ⁿ each will be generated, with the proviso that if the polymorphic genomic region is haploid or hemizygous in the individual (e.g., if it from a sex-linked, mitochondrial or chloroplast gene), an evidence score of 1 is assigned.

(3) The frequency scores for each haplotype are summed across all the samples to yield the initial haplotype frequency. For example, one haplotype may occur in the expansions of two genotypes, one singly heterozygous and on doubly heterozygous. The total initial frequency for this haplotype from the two genotypes would then be 1 plus 0.5, or 1.5. Where multiple identical genotypes have been grouped together, the evidence scores for haplotypes associated with that genotype are multiplied by the group multiplier n_{g} to simultaneously account for all occurrences of that genotype. The total initial frequency, added up across all haplotypes, will be two times the number of samples if all genomic regions are diploid; if haploid or hemizygous regions are represented the total will be reduced accordingly.

In the Hap Expansion phase, the evidence score is a function of the number of ambiguous polymorphic sites being haplotyped. As used herein, an ambiguous polymorphic site means either a heterozygous site or is a site for which nucleotide sequence information is lacking. Preferably, the evidence score sᵢ obeys one or both of the following formulas:

$$0 \leq {s}_{i} \leq 2 ; and {\sum }_{i = 1}^{n} {s}_{i} = 2 ;$$

wherein n is the number of ambiguous positions being haplotyped in the genotype. In a current preferred embodiment exemplified herein, the evidence score sᵢ is =2/2ⁿ, wherein n is the number of ambiguous positions being haplotyped in the genotype. In each of the above embodiments, however, if the polymorphic genomic region is haploid or hemizygous, an evidence score of 1 is assigned.

Also, preferably, the initial frequency fᵢ is calculated from the sum of the evidence scores across all the different individuals (or genotypes, where the evidence scores sᵢ are weighted appropriately by multiplying by the group multipliers n_{g}), for each of the enumerated possible haplotypes hᵢ, wherein it is understood that hᵢ is an index, e.g., hᵢ, hⱼ, etc.

An example of the assignment of evidence scores for the fictitious NoName gene, having four polymorphic sites, is illustrated in Table 1. Such a gene would have a total of 3⁴=81 possible genotypes, and 2⁴=16 possible haplotypes. Haplotype expansions of four genotypes, from a population of four different individuals, are illustrated. Table 1 shows all possible haplotypes which could be enumerated from each of the four illustrated genotypes, one of which is heterozygous at each site (16 haplotypes), two which are heterozygous at only 3 of these sites (8 haplotypes) and one of which is heterozygous at only 2 of these sites (4 haplotypes). The set of evidence scores sᵢ for each genotype, and the derived initial frequency scores fᵢ for each haplotype summed across all four genotypes, are also shown. Table 2 shows similar information for the same haplotypes, but where two additional individuals having genotype 2 have been added to the population, and illustrates an embodiment of the invention wherein grouping of identical genotypes has been carried out. In this embodiment, the evidence scores for genotype 2 are multiplied by n_{g} (in this example, n_{g}=3), prior to calculation of the frequency scores fᵢ.

TABLE 1
Assignment of evidence scores sᵢ and initial frequency scores fᵢ
for all possible haplotypes expanded from four genotypes of the NoName Gene.

| | Genotype1 | | | | | Genotype 2 | | | | | | | | | | | | | | | |
|---|---|---|---|---|---|---|---|---|---|---|---|---|---|---|---|---|---|---|---|---|---|
| | | A/ | | | | | A/ | | | | Genotype 3 | | | | | Genotype 4 | | | | | |
| hᵢ | A/G | C | C/T | G/T | sᵢ | A/A | C | C/T | G/T | sᵢ | A/G | C/C | C/T | G/T | sᵢ | G/G | A/C | T/T | G/T | sᵢ | fᵢ |
| 1 | A | A | C | G | 0.125 | A | A | C | G | 0.250 | | | | | | | | | | | 0.375 |
| 2 | A | A | C | T | 0.125 | A | A | C | T | 0.250 | | | | | | | | | | | 0.375 |
| 3 | A | A | T | G | 0.125 | A | A | T | G | 0.250 | | | | | | | | | | | 0.375 |
| 4 | A | A | T | T | 0.125 | A | A | T | T | 0.250 | | | | | | | | | | | 0.375 |
| 5 | A | C | C | G | 0.125 | A | C | C | G | 0.250 | A | C | C | G | 0.250 | | | | | | 0.625 |
| 6 | A | C | C | T | 0.125 | A | C | C | T | 0.250 | A | C | C | T | 0.250 | | | | | | 0.625 |
| 7 | A | C | T | G | 0.125 | A | C | T | G | 0.250 | A | C | T | G | 0.250 | | | | | | 0.625 |
| 8 | A | C | T | T | 0.125 | A | C | T | T | 0.250 | A | C | T | T | 0.250 | | | | | | 0.625 |
| 9 | G | A | C | G | 0.125 | | | | | | | | | | | | | | | | 0.125 |
| 10 | G | A | C | T | 0.125 | | | | | | | | | | | | | | | | 0.125 |
| 11 | G | A | T | G | 0.125 | | | | | | | | | | | G | A | T | G | 0.500 | 0.625 |
| 12 | G | A | T | T | 0.125 | | | | | | | | | | | G | A | T | T | 0.500 | 0.625 |
| 13 | G | C | C | T | 0.125 | | | | | | G | C | C | T | 0.250 | | | | | | 0.375 |
| 14 | G | C | C | G | 0.125 | | | | | | G | C | C | G | 0.250 | | | | | | 0.375 |
| 15 | G | C | T | G | 0.125 | | | | | | G | C | T | G | 0.250 | G | C | T | G | 0.500 | 0.875 |
| 16 | G | C | T | T | 0.125 | | | | | | G | C | T | T | 0.250 | G | C | T | T | 0.500 | 0.875 |

| | | | | | |
|---|---|---|---|---|---|
| Totals | 2.000 | 2.000 | 2.000 | 2.000 | 8.000 |

TABLE 2
Assignment of evidence scores sᵢ and initial frequency scores fᵢ
for all possible haplotypes expanded from four genotypes of the NoName Gene; with grouping of genotype 2.

| | Genotype1 | | | | | Gentype 2 (3 occurrences) | | | | | Genotype 3 | | | | | Genotype 4 | | | | | |
|---|---|---|---|---|---|---|---|---|---|---|---|---|---|---|---|---|---|---|---|---|---|
| | | | C/ | | | | | C/ | | | | | C/ | | | | | T/ | | | |
| hᵢ | A/G | A/C | T | G/T | sᵢ | A/A | A/C | T | G/T | (n_{g})(sᵢ) | A/G | C/C | T | G/T | sᵢ | G/G | A/C | T | G/T | sᵢ | fᵢ |
| 1 | A | A | C | G | 0.125 | A | A | C | G | 0.750 | | | | | | | | | | | 0.875 |
| 2 | A | A | C | T | 0.125 | A | A | C | T | 0.750 | | | | | | | | | | | 0.875 |
| 3 | A | A | T | G | 0.125 | A | A | T | G | 0.750 | | | | | | | | | | | 0.875 |
| 4 | A | A | T | T | 0.125 | A | A | T | T | 0.750 | | | | | | | | | | | 0.875 |
| 5 | A | C | C | G | 0.125 | A | C | C | G | 0.750 | A | C | C | G | 0.250 | | | | | | 1.125 |
| 6 | A | C | C | T | 0.125 | A | C | C | T | 0.750 | A | C | C | T | 0.250 | | | | | | 1.125 |
| 7 | A | C | T | G | 0.125 | A | C | T | G | 0.750 | A | C | T | G | 0.250 | | | | | | 1.125 |
| 8 | A | C | T | T | 0.125 | A | C | T | T | 0.750 | A | C | T | T | 0.250 | | | | | | 1.125 |
| 9 | G | A | C | G | 0.125 | | | | | | | | | | | | | | | | 0.125 |
| 10 | G | A | C | T | 0.125 | | | | | | | | | | | | | | | | 0.125 |
| 11 | G | A | T | G | 0.125 | | | | | | | | | | | G | A | T | G | 0.500 | 0.625 |
| 12 | G | A | T | T | 0.125 | | | | | | | | | | | G | A | T | T | 0.500 | 0.625 |
| 13 | G | C | C | T | 0.125 | | | | | | G | C | C | T | 0.250 | | | | | | 0.375 |
| 14 | G | C | C | G | 0.125 | | | | | | G | C | C | G | 0.250 | | | | | | 0.375 |
| 15 | G | C | T | G | 0.125 | | | | | | G | C | T | G | 0.250 | G | C | T | G | 0.500 | 0.875 |
| 16 | G | C | T | T | 0.125 | | | | | | G | C | T | T | 0.250 | G | C | T | T | 0.500 | 0.875 |

| | | | | | |
|---|---|---|---|---|---|
| Totals | 2.000 | 6.000 | 2.000 | 2.000 | 12.000 |

C. HAP ASSIGNMENT

The haplotype frequency scores fᵢ generated in the Hap Expansion serve as an initial estimate of the expected frequency of the haplotypes. Many genotypes will allow only one possible combination of two of the haplotypes from the expansion. This is true for the homozygous and singly heterozygous genotypes. For multiply heterozygous genotypes, there are generally many possibilities. However, since of the 2ⁿ theoretically possible haplotypes only ˜n actually occur, many real haplotypes will occur in more than one of the samples, and their frequency scores fᵢ will be higher than those of rare or non-occurring haplotypes. A pair frequency score Fₖ can be assigned to each pair of haplotypes. The pair frequency score Fₖ is a function of the haplotype frequency scores (fᵢ, fⱼ) for each of the haplotypes hᵢ and hⱼ in the pair. In a preferred embodiment, only haplotypes that meet a frequency score criterion are considered when assigning the pair frequency score Fₖ. The frequency score criterion may be user defined or it may be a default value. In a particularly preferred embodiment, the frequency score criterion is set at fᵢ>0.1. This means that haplotypes with less than a 10% chance of occurring in any individual in the entire sample are eliminated from the Hap Assignment phase. Lower values for the frequency score criterion (e.g., fᵢ>0.01, or fᵢ>0.001) will result in slightly greater accuracy in making hap pair assignments, but greater computing time and/or resources will be required for lower values. Thus, the value for this criterion may be any number that the practitioner skilled in the art might find suitable to balance the desired degree of accuracy with the constraints on available time and computational resources.

In the Hap Assignment phase, the pair score criterion is preferably (a) a specific numerical cutoff; (b) a function of the values of the pair scores; or (c) a function of the rankings of the pair scores. In a preferred method of the invention exemplified herein, each pair (i, j)ₖ of haplotypes hᵢ and hj in a sample is assigned a pair score Fₖ=2fᵢfⱼ, if i≢j; or Fₖ=fᵢ² if i=j; with the proviso that F_{k=f}ᵢ when the polymorphic genomic region is haploid or hemizygous.

The Hap Assignment phase of the method of the invention preferably further comprises determining a probability pₖ that the haplotype pair which has been assigned to the genotype is correct. In one embodiment of the invention, the probability score pₖ is determined by (a) ranking each of the pair scores Fₖ for all the N possible pairs for a genotype with the highest score (F₀) first; and (b) defining the probability pₖ as the score of the first pair divided by the sum of all scores:

$${p}_{k} = \frac{{F}_{k}}{{\sum }_{l = 0}^{N} {F}_{l}} .$$

Under these criteria, if there are two equally likely assignments (e.g., F₀=36, F₁=36), a score of 0.5 is given to the assignment, reflecting the fact that we are only 50% certain that one of these pairs is the correct one. As another example, if there is one pair with score 8, and one with score 2, the assignment of the first pair is made with an 80% probability. The ranking is performed this way for all the genotypes.

In a preferred embodiment of the invention, which is exemplified herein, the probability score pₖ is determined by (a) ranking each of the pair scores Fₖ for all the possible pairs for a genotype with the highest score (F₀) first; (b) disregarding all but the Nᵣₐₙₖ highest ranking assignments; and (c) defining the probability pₖ as the score of the first pair divided by the sum of all scores, using the formula:

$${p}_{k} = \frac{{F}_{k}}{{\sum }_{l = 0}^{{N}_{rank}} {F}_{l}} .$$

D. ITERATION

In the method of the present invention, a revised haplotype frequency score fᵢ is calculated for each of the haplotypes, wherein the revised haplotype frequency fᵢ is a function of the previously determined probability pₖ for each consistent haplotype pair which contains the haplotype. In a method of the invention exemplified herein, a new set of frequency estimates is calculated from the probability scores. The new frequency fᵢ of haplotype i is calculated as the sum of the pₖ for all pair assignments containing haplotype i, counting homozygous pairs (i, i) twice. Again, the sum of the frequencies across all haplotypes will be two times the number of samples, if all genomic regions are diploid; if haploid or hemizygous regions are represented the total will be reduced accordingly.

Revised haplotype pair scores Fₖ and revised probability scores pₖ can be determined based on the revised haplotype frequency scores fᵢ using the methods described above. These steps can be repeated until an end condition is reached.

In a preferred embodiment, where identical genotypes have been grouped together prior to Hap Expansion, the new frequency fᵢ of haplotype hᵢ is calculated as the sum of the products (n_{g})(pₖ) for all pair assignments containing haplotype hᵢ, further multiplied by 2 for homozygous pairs (i, i).

The end condition can be one of many possible parameters. It may be user definable or a default condition. It may also be variable or set. For example, the end condition may be met when the above-mentioned iteration steps are repeated a preset number of times. Alternatively, the end condition may be met when one or more of the parameters fᵢ, Fₖ, and pₖ stabilizes; or the end condition may be met simply when the operator chooses to stop.

Stabilization can mean: (1) the maximum difference between consecutive iterations of Fₖ or pₖ goes below a threshold; (2) the ranking (or truncated ranking) does not change for a given number of iterations, or (3) any suitable quantity does not change more than a threshold. The preferred end condition is (1). In another preferred method of the invention exemplified herein, the end condition is met when the operator chooses to stop.

E. AMBIGUITY CRITERION AND PAIR SCORE CRITERION

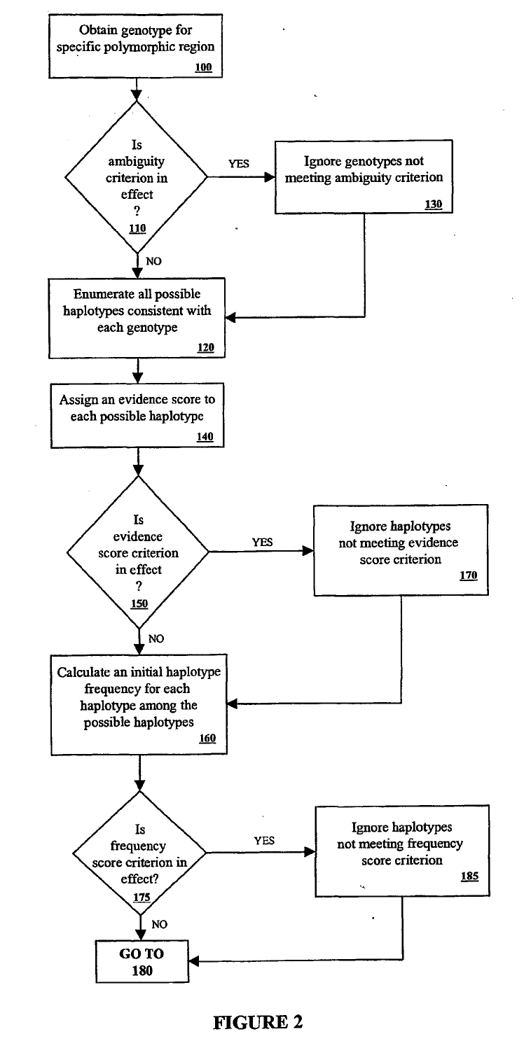

In one embodiment of the invention, only genotypes that meet an ambiguity criterion have their haplotypes enumerated (see FIG. 2). The ambiguity criterion may be user definable or may be a default value. The ambiguity criterion is preferably a function of the number of ambiguous polymorphic sites in the genotype, wherein an ambiguous polymorphic site is either a heterozygous site or is a site for which information is lacking.

In another aspect of the invention, only haplotype pairs that meet a pair score criterion will be kept. The pair score criterion is a function of the pair score Fₖ for each of the haplotype pairs, as discussed above. In an embodiment of the invention described herein, the pair score Fₖ is retained only if it is one of the top 15 pair scores. In another embodiment, only those haplotype pairs whose pair scores Fₖ are greater than a certain percentage of Fₖmax (the highest Fₖ associated with any consistent haplotype pair) will be kept.

F. ERROR DETECTION AND CORRECTION

At all stages in the method, the possibility of error in the input data may be considered. In the expansion, for example, even a fully homozygous sample may generate a number of haplotypes, i.e., the principal one, and all the additional ones which would be introduced if any one of the positions were changed to be heterozygous. In the method of the invention exemplified herein, the score of each such additional haplotype is considerably reduced by multiplying by the assumed error probability, a number usually set at 0.01-0.02. At the assignment stage, if the most highly scoring haplotype pair is not consistent with the input genotype (despite the strong 1%-2% penalty factor), the difference is highlighted and reported as a probable misread (e.g., a sequencing error).

The preferred way to modify the method to allow for the possibility of errors is as follows. For each measured genotype (i.e. for each individual at each polymorphic position), replace the exclusive determination (either of A, A/C, C) by the specification of probabilities as follows: p_{c} probability for the common allele, pₕ for the heterozygote, and pᵣ for the rare allele. Preferably, these probabilities are estimated individually for each genotype measurement according to the quality of the raw data by the procedure used to determine the genotypes. Alternatively, a single error probability pₑᵣᵣ can be defined that estimates the probability for any given allele to be determined erroneously. In the latter case the genotype probabilities are as follows: (a) For a measured homozygous common allele: p_{c}=(1−pₑᵣᵣ)², pₕ=2(1−pₑᵣᵣ)pₑᵣᵣ, pᵣ=pₑᵣᵣ²; (b) for a measured heterozygote: p_{c}=½pₑᵣᵣ, pₕ=1−pₑᵣᵣ, pᵣ=½pₑᵣᵣ; and (c) for a measured homozygous rare allele: p_{c}=pₑᵣᵣ², pₕ=2(1−pₑᵣᵣ)pₑᵣᵣ, pᵣ=(1−pₑᵣᵣ)². Preferably a more complex formula involving the allele frequencies would be used. In step (b) of the Hap Expansion process described earlier, each haplotype may be individually weighted for its consistency with a given individual genotype. The preferred weights for the Hap Expansion are

$${w}_{i} = {\prod }_{k = 1}^{N} {c}_{ik} ( {p}_{c} + \frac{{p}_{h}}{2} ) + {r}_{ik} ( {p}_{r} + \frac{{p}_{h}}{2} ) \qquad ( Formula 1 )$$

where k=1 . . . N enumerates the polymorphic sites, Cᵢₖ=1, rᵢₖ=0 if the allele of haplotype hᵢ at position k is the common allele, and cᵢₖ=0, rᵢₖ=1 if it is the rare allele. The score sᵢ is then simply multiplied by the weight so that the weighted score sᵢ′=wᵢsᵢ.

In step (f) of the Hap Assignment process, a pair of haplotypes hᵢhⱼ may be weighed for its consistency with an individual genotype. The preferred weights for the Hap Assignment are

$${w}_{ij} = {\prod }_{k = 1}^{N} {c}_{ik} {c}_{jk} {p}_{c} + ( {c}_{ik} {r}_{jk} + {r}_{ik} {c}_{jk} ) {p}_{h} + {r}_{ik} {r}_{jk} {p}_{r} . \qquad ( Formula 2 )$$

Step (f) is then modified such that the Fₖ are replaced by Fₖ′=wᵢⱼFₖ (recall that k stands for a pair of haplotypes (hᵢhⱼ)). With these modifications, it is possible for a pair of haplotypes which is inconsistent with the observed genotype to nevertheless score high in the ranked list of assignments.

Two cases may be distinguished: Case 1: An inconsistent pair has a score that is comparable to, but lower than, the score of a consistent pair. In this case, one may conclude that there is a significant probability that the genotype causing the inconsistency was measured incorrectly. In the presently implemented version of the invention, this genotype is characterized as a possible miscall (error detection). Case 2: An inconsistent pair ranks first on the list of possible assignments. In the presently implemented version of the invention, this genotype is characterized as most likely wrong, and we choose the haplotype assignment in spite of its inconsistency, effectively overriding the genotyping call (error correction).

G. PRUNING

In one embodiment of the invention, a recursive pruning algorithm is used in the Hap expansion phase to eliminate from consideration enumerated haplotypes whose evidence scores sᵢ are below a given threshold value, and/or is used in the Hap assignment phase to eliminate from consideration haplotype pairs whose pair scores Fₖ are below a threshold value. A pruning algorithm is preferred because the number of possible haplotypes grows exponentially with the number of sites, and because an exhaustive enumeration is rarely desirable.

Since the weights wᵢ and wᵢⱼ are written as products across the sites in the above formulas, they can be recursively enumerated as follows: (a) generate the two alleles for the first position (the first polymorphic position chosen to be included in haplotyping); (b) calculate the first factor of the weight, i.e., evaluate formula 1 or formula 2 with k=1; (c) for each of the first position alleles, generate the two alleles at the second position; (d) for each combination of alleles, calculate the second factor of the weight and multiply it by the first factor, i.e., evaluate formula 1 or formula 2 for k=(1, 2); (e) do not continue with combinations where the weight is below a given threshold; and (f) continue generating additional combinations, one site at a time, until all positions have been visited. The threshold for wᵢ is an evidence score criterion, and the threshold for wᵢⱼ is a pair score criterion.

In other words, one generates sub-haplotypes with one, two, three, up to the full set of polymorphic sites. One generates a new set of subhaplotypes from the previous set of subhaplotypes by creating the two possible combinations for each of the n^{th} subhaplotype with either of the two alleles for the (n+1)^{th} polymorphic site. The net result is that the weights are recalculated and the ambiguity threshold is re-tested as each polymorphic position is considered in turn. Whenever a subhaplotype containing multiple rare polymorphisms is encountered, the weight of all haplotypes comprising that subhaplotype are necessarily reduced below the threshold, indicating that all haplotypes comprising that subhaplotype need not be visited. Without the pruning step (e), for some genotypes, many possible haplotypes would be generated, most of them having vanishing weight. Step (e) ensures that any branches of the search that are already doomed because of too many mismatches and/or rare polymorphisms will not be followed. This changes the computational complexity of the algorithm such that it rises more or less linearly rather than exponentially with the number of sites, making the calculation more practical.

In a preferred embodiment, the evidence score criterion is chosen to optimize the use of computer resources. The evidence score criterion s_{trunc} is a score threshold, below which the recursive search for consistent haplotypes based on the current sub-haplotype is truncated. It must be less than 1, and should be as small as the time available for computation allows. It is related to the number nₐ of ambiguous polymorphic sites allowed in a genotype, beyond which no more contributions to the hap expansion will be generated, in the following way: S_{trunc}=(½)ⁿ^{ₐ}. If the number of samples is N, the maximum frequency a haplotype could have in the population and escape consideration is (N)(s_{trunc}). A preferred value for the threshold is therefore s_{trunc}<1/N. In a preferred embodiment s_{trunc}=0.01. As alternative examples, s_{trunc} may be 0.001, 0.0001, or 0.00001, or any other number that the practitioner skilled in the art might find suitable in view of the constraints on available time and computational resources.

H. MENDELIAN INHERITANCE

Another aspect of the method of invention provides a method for optionally adjusting the assignment probability scores pₖ to reflect the requirement of Mendelian inheritance between individuals who are related. For example, when there is at least one multi-generation family included among the individuals whose polymorphic genomic regions are being haplotyped, the probability pₖ may be reduced for each pair assignment for each genotype in the family that does not obey Mendelian inheritance. In the simplest embodiment of this aspect of the invention, the scores for any assignment which does not obey Mendelian inheritance with respect to other higher ranking assignments for the relatives are set to zero. Another, preferred, embodiment is the multiplication of an unadjusted probability score p^{o}ₖ by (1−pₖ′), where pₖ′ is the score of any assignment k′ of a related person that is in conflict with the first assignment k (i.e., pₖ=p^{o}ₖ(1−pₖ′), where pₖ′ is the probability calculated for a pair assignment of a related genotype). Another embodiment, which is currently implemented in the example described herein, is the interactive fixing of selected assignments (by setting pₖ=1) according to the judgment of the operator. The scores are renormalized after such modification by using the formula

$${p}_{k} = \frac{{F}_{k}}{{\sum }_{l = 0}^{{N}_{rank}} {F}_{l}} . \qquad ( Formula 3 )$$

In other words, if some of the samples come from individuals that are related, Hap assignments are preferably constrained to obey Mendelian segregation rules, i.e. one of the copies must be inherited from the father, and one from the mother. This constraint is used in the HAP™ Builder process to eliminate solutions that violate inheritance rules and increase the probability scores of those that do not. It will be appreciated that individuals who in fact are not the offspring of an erstwhile parent are readily identified, and their haplotype pair assignments will not be subjected to the Mendelian segregation criterion.

Mendelian segregation rules can also be used to validate the HAP™ Builder process. For example, genotype information from individuals belonging to one or more three-generation families may be entered into the database so that they can be treated as either being related or not being related. The haplotype assignments under each of these conditions can be compared for consistency.

I. HARDY-WEINBERG EQUILIBRIUM

Another aspect of the method of the invention provides for the optional adjustment of the assignment probability scores pₖ to reflect Hardy-Weinberg Equilibrium. In any given Mendelian population, the number of heterozygotes and homozygotes are related by the Hardy-Weinberg principle: f₀₀=p², f₀₁=2pq, and f₁₁=q², where f₀₀ is the frequency of wild type homozygotes, f₀₁ the frequency of heterozygotes, and f₁₁ the frequency of mutant homozygotes. P and q are the frequencies of the wild type and mutant alleles, respectively. This is true for individual polymorphisms, and can also be extended for haplotypes (fᵢᵢ=pᵢ², fᵢⱼ=2pᵢpⱼ). Since we observe three variables (or n+n(n−1)/2), which reduce to just 2 (or n), the Hardy-Weinberg principle gives us an additional constraint which is used in the HAP™ Builder process to increase the probability scores of those haplotypes which satisfy the Hardy-Weinberg principle, and decrease the scores of those that do not.

When there is at least one population group included among the individuals whose polymorphic genomic regions are being haplotyped and whose genotypes would be expected to reflect Hardy-Weinberg Equilibrium, the probability pₖ may be reduced for each pair assignment for each genotype in the population group that does not obey Hardy-Weinberg Equilibrium.

The Hardy-Weinberg equilibrium postulates a relationship between the frequencies of homozygous assignments and heterozygous assignments such that

Fᵢᵢ²+2Fᵢⱼ+Fⱼⱼ²=1, (Formula 4)

where the Fᵢᵢ, Fⱼⱼ, and Fᵢⱼ are the frequencies Fₖ of the three possible assignments for any given pair of different haplotypes hᵢ and hⱼ. One embodiment of this score adjustment is to multiply the scores pᵢᵢ, pⱼⱼ, and pᵢⱼ by one minus the Xi squared value for the deviation from Hardy-Weinberg equilibrium for all pairs of different haplotypes hᵢ and hⱼ:

$${p}_{k}^{′} = {p}_{k} ( 1 - \frac{{( {F}_{il} - {f}_{i}^{2} )}^{2} + {( {F}_{ij} - 2 {f}_{i} {f}_{j} )}^{2} + {( {F}_{jj} - {f}_{j}^{2} )}^{2}}{{f}_{i}^{4} + 4 {f}_{i}^{2} {f}_{j}^{2} + {f}_{j}^{4}} ) \qquad ( Formula 5 )$$

In other words, when a haplotype pair does not fit the Hardy-Weinberg equation, the probability pₖ may be reduced to p′ₖ by the above formula, wherein fᵢ, and fⱼ are the frequencies of haplotypes hᵢ and hⱼ in the population group and Fᵢᵢ, Fⱼⱼ and Fᵢⱼ are the frequencies of each possible pair of haplotypes hᵢ and hⱼ in the population group.

J. INFERRING HAPLOTYPES AT AMBIGUOUS SITES

Another aspect of the invention provides methods and tools to infer haplotypes from every genotype, despite the presence of ambiguous polymorphic sites (sites where data is absent). As described elsewhere herein, the input to the program for each genotype measurement is a set of three probabilities, one each for a homozygous common allele, for a heterozygote and for a homozygous rare allele. If no data is available at all, in the HAP™ Builder program as currently implemented these probabilities default to 0.25, 0.5, and 0.25, respectively. The program accommodates these probabilities and still generates the most likely haplotype pair assignments. The missing genotypes can then be inferred by combining the appropriate alleles from the assigned pair of haplotypes.

K. END CONDITIONS

The iterative calculation and re-calculation of pair scores, probability scores, and haplotype frequency scores leads to convergence of these values toward certain limits. It is not necessary to allow these limits to be reached in order to make use of the invention, since at some point the assignments of haplotypes and haplotype pairs that the practitioner can make based on the scores will not be altered by further iterations. For this reason the practitioner of this invention may specify end conditions that will trigger the termination of iterations by the HAP™ Builder program. Alternatively, the practitioner may use his or her own judgment and terminate the iterations at will when the iterations have produced a satisfactory result.

Examples of suitable end conditions are:

(a) the values of all fᵢ, pₖ, and/or Fₖ in consecutive iterations differ by less than a preselected amount, or differ by less than a preselected percentage,

(b) a preset number of iterations have been carried out,

(c) the rank order of the Fₖ for the haplotype pairs under consideration does not change in consecutive iterations.

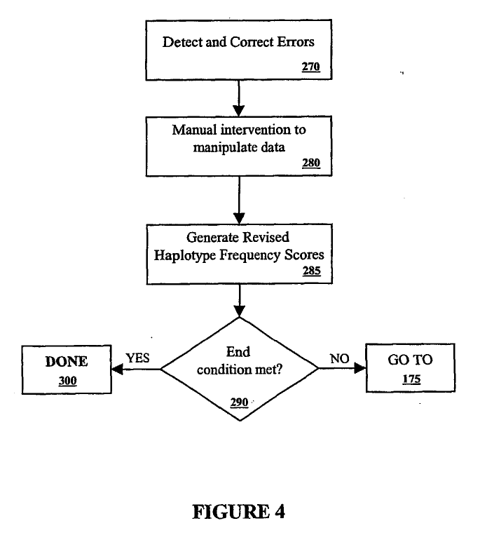

In the particular embodiment exemplified herein, an end condition is tested for after a new set of haplotype frequency scores have been iterated (see FIG. 4). However, it will be apparent that an end condition can be tested for at any point during the iterations, and such alternative embodiments are considered part of the invention. For example, if the end condition is a function of the pair scores, it will be appropriate to test for the end condition after a new set of pair scores have been iterated. Where operator intervention, based upon human judgment, is the means for terminating the iterations, the iterations can of course be ended at any point.

L. SUMMARY OF THE MAJOR ADVANTAGES OF PREFERRED EMBODIMENTS OF THE INVENTION

1) The use of cut-offs to speed up the process. This happens in one or several places: (a) the Hap expansion only considers contributions down to a certain probability cut-off, (b) only a limited number of haplotype pair assignments are kept, and (c) haplotypes whose frequency falls below a certain frequency threshold are dropped from the next iteration. Pruning is the preferred way to effect cutoffs (a) and (b).

2) The assignment of “competitive quality scores” based on the ranked list of possible assignments. This accounts well for ambiguous calls, where the score will be close to 0.5 because there are two equally likely solutions to the problem.

3) The “error detection and correction” aspect. Each genotype is never assumed to be just one that was measured, but could be any with weighted probability. For example, an A is not just an A, it is, an A with a probability of (1−p)², an A/G with probability of 2p(1−p), or even a G with a (vanishing) probability of p². Here, p is the “error probability”, and is usually close to 0. A value of 0.01 is employed in the current preferred embodiment, corresponding to a probably exaggerated) accuracy of 99% in calling the genotypes. In the Hap expansion, these probabilities are used as weights, so that, for example, the genotype A G C/T, which would normally expand into the haplotype pair AGC+AGT, may expand into something like this:

0.94(AGC+AGT)+0.01(AGC+CGT)+0.01(CGC+AGT)+. . .

Obviously, there are a very large number of possibilities, most of them with very small weights. A simple recursive pruning method is used to find all the contributions above a certain threshold weight, currently set at 0.01, such that only single error possibilities are used. A similar pruning algorithm is used for the haplotype pair assignment, where assignments are made that do not exactly fit the genotype, with the appropriate low weight.

4) Integration of family data, Hardy-Weinberg equilibrium.

Family transmission and Hardy-Weinberg equilibrium may be checked as part of the iteration procedure, which should increase the accuracy of the calls even for those assignments made for individuals that are not related.

IV. EXAMPLE

Genotype data for input into the HAP™ Builder program may be generated by the practitioner by sequencing DNA from a population of interest, or may be obtained from various commercial sources of genotype data such as commercial SNP database providers. Publicly available SNP databases may also be used, such as for example the Human Genic Bi-Allelic Sequences database (HGBASE), the dbSNP database maintained by the National Center for Biotechnology Information, and the Human SNP database maintained by the Whitehead Institute at the Massachusetts Institute of Technology. These public databases are readily accessible via the internet. The data is suitably formatted when stored in a DecoGen™ database as described in U.S. application Ser. No. 60/141,521, filed Jun. 25, 1999, and international application WO 01/01218, which are incorporated herein by reference.

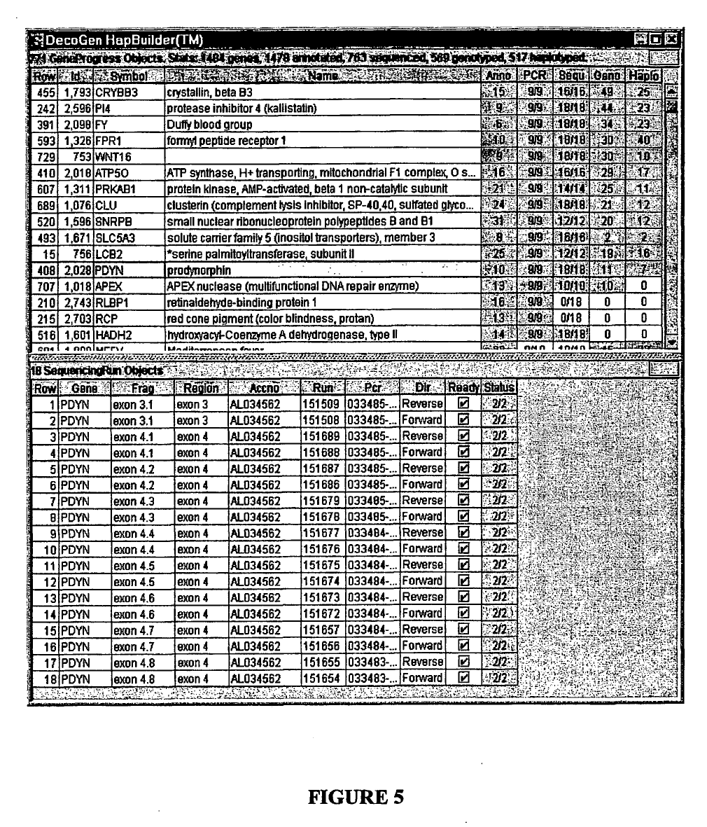

In the present invention, a person may use a user terminal to view a screen which allows the user to see all of the candidate genes, or a subset thereof, and to bring up further information. This screen (as well as all the other screens described herein) may, for example, be presented as a web page, or a series of web pages, from a web server. This web based use may involve a dedicated phone line, if desired. Alternatively, this screen may be served over the network from a non-web based server or may simply be generated within the user terminal. An example of such a screen referred to herein is illustrated in the top half of FIG. 5.

The top half of FIG. 5 is an example of a screen showing a set of candidate genes for which polymorphism data has been obtained or is in the process of being obtained. This polymorphism data and other information described below may be stored in a database such as the one described in U.S. application Ser. No. 60/141,521 and in international application WO 01/01218, or is calculated from information stored in such a database. Most of the information shown in later figures is specific to the Index Repository described herein.

The screen shows genes for which data is currently available in a database useful in the invention and those queued for processing (and for which data will appear in the database). The “Row” column indicates the order in which genes were entered into the database, while the “Id” column is a numerical identifier for the gene having the symbol and name indicated in the “Symbol” and “Name” columns. The columns on the right side of the screen indicate various stages in the process of analyzing target regions of the gene identified in the corresponding row. For example, “Anno” is shorthand for “Annotation”, which is the operation performed at the beginning of the gene analysis process to annotate different features of the gene structure, such as the locations and sequences of the promoter, exons and introns as described in more detail below. The number in the Anno column provides the number of different annotated features of the gene. The “PCR” and “Sequ” columns indicate how many of the target regions of the gene have been analyzed successfully by the PCR and Sequencing production groups, respectively. The number of polymorphic sites identified for the gene is shown in the “Geno” column. Similarly, the number of haplotypes deduced by the HAP™ Builder method of the present invention is shown in the “Haplo” column. The various colors provide an immediate visual indicator of the status of the gene at each stage of analysis, with green and yellow indicating completely done and in progress, respectively, and white indicating no target regions have arrived to that stage in the analysis process. Alternatively, the status of genes in the different production stages may be indicated by different degrees or types of shading. The genes in the database may be sorted by various criteria by clicking on any of the columns shown in the top half of FIG. 5, e.g., clicking on “Id” allows the genes to be sorted in ascending or descending numerical order, clicking on “Name” allows the genes to be sorted in alphabetical order, and clicking on “Sequ” allows the genes to be sorted by number of fragments.

The user can select a gene to examine in detail by using the mouse (or other user-input device such as keyboard, roller ball, voice recognition, etc.) to select the candidate gene. In the example depicted in FIG. 5, the prodynorphin gene is selected, as indicated by the purple color in what is shown in Row 408 of the figure. The screen may optionally include a “find” feature, to locate a candidate gene of interest. In the exemplified screen, a single click on the selected gene brings up the screen shown in the bottom half of FIG. 5, which provides sequencing wordflow information, i.e., numerical workflow identifiers for the sequencing and PCR reactions (“Run” and “PCR” columns), in both forward and reverse directions (“Dir” column), that have been performed for various fragments from each of the target regions of the gene (for example, fragment exon 3.1 from exon 3). A check in the “Ready” column indicates when a gene fragment is ready to be analyzed for polymorphisms and the “Status” column indicates whether there is sequencing information for both strands of the fragment. Such information and screens are not necessary for using the methods of the present invention, but may be used to monitor the progress and/or extent of sequencing of candidate gene(s) (or other loci) input into the database and may be useful in providing an estimate of the reliability of the sequence data which has been input into the database. Decisions about whether or not to proceed with polymorphism analysis in one or more of the fragments of the selected gene may be based on the status of the sequencing runs. For example, if sequence information is available for both strands, the more reliable the sequence will be and, therefore, the more reliable the polymorphism data will be.

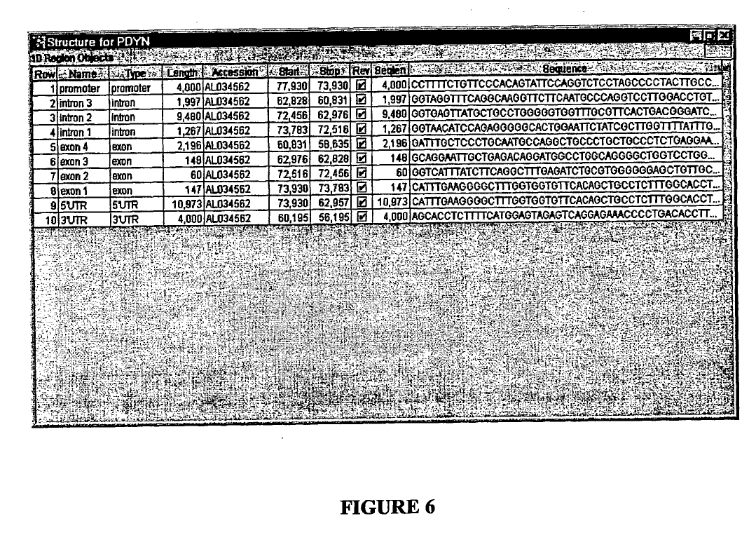

FIG. 6 shows an example of the annotation screen, which is reached by clicking on “Anno” in the screen depicted in FIG. 5. As indicated in FIG. 6, the PDYN gene contains 10 features, each of which has the indicated lengths and the indicated start and stop positions with respect to the indicated Accession number. The Accession number is typically the GenBank Accession number for the gene, although it may be an identifying number from another publically available database or an internal identifying number. If the complete gene sequence is not know, the “Accession” column may contain multiple identifying numbers for partial sequences. A check in the “Rev” column indicates the coding sequence for the gene is found in the reverse complement of the Accession number. The “Seqlen” column indicates the number of nucleotides entered into the “Sequence” box at the end of the row. The amount of sequence shown may be increased by enlarging the window; the entire sequence for a feature may be displayed by clicking on the particular sequence of interest. The information contained in the “Anno” screen is typically derived from GenBank and other public data sources.

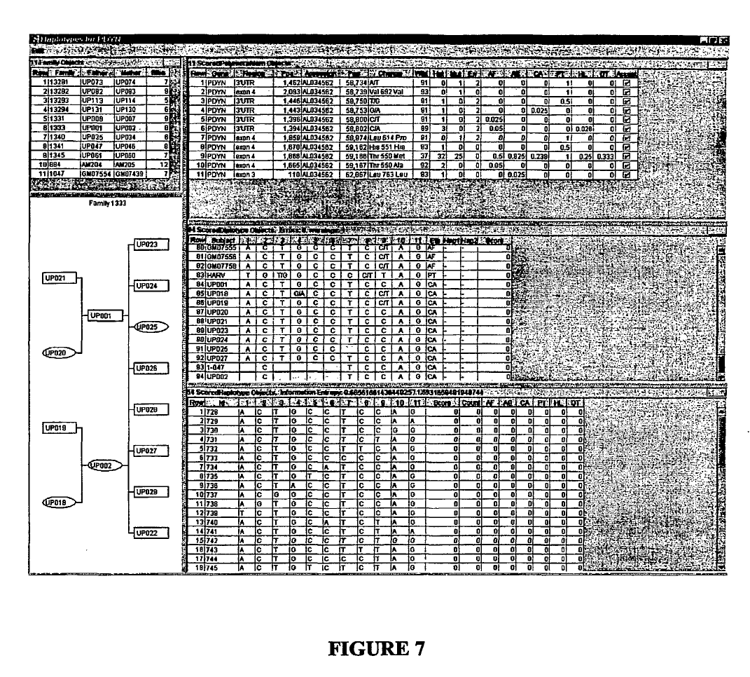

In the screen exemplified in FIG. 5, a single click on the haplotype (“Haplo”) column in that row brings up the screen for the HAP™ Builder program, an example of which is shown in FIG. 7.

The screen exemplified in FIG. 7 shows several boxes at the same time, although one or more of the boxes may be expanded by dragging the dividers between the boxes. The window on the left (labeled “Family Objects” in FIG. 7) will typically show a list of the different multi-generation families available for polymorphism analysis and relevant information concerning each family, such as numerical identifiers for the father and mother, and the number of children “siblings”. This window will typically show a family tree below the list of families. Males are shown as rectangular boxes and females are shown as ovals. Family 1333 is selected in the box on the upper left side, therefore, the family tree for that family is displayed. Family trees for other families may be displayed by clicking on the name of the desired family in the top of the window. If nothing had been clicked on, Family 13291 would have been the default family tree displayed.

The screen exemplified in FIG. 7 will typically also show a box that provides information about the polymorphism data for a selected gene (labeled “ScoredPolymorphism Objects” in top right side of FIG. 7). Each row contains information for a different polymorphic site (PS) identified in the gene from a population (a group of people whose nucleotide sequences have been examined for this gene). In this example, the screen indicates that eleven PS were detected in the PDYN gene. The “Region” column indicates the region in the gene where the polymorphic site is located (e.g., the promoter, the first intron, the first exon, etc.). The number in the first “Pos” column indicates the location of the polymorphism in the indicated region of the gene, while the number in the second “Pos” column indicates the location of the polymorphism in the genomic sequence, based on the numbering of the Accession sequence. The Accession number is preferably the same Accession number as presented in the “Anno” screen, although it may be a different number. The rows can be sorted by clicking on “Row”, “Position” or “Accession”. Clicking on “Row” orders the gene from 5′ to 3′. The “Change” column typically contains the identity of the alternative nucleotides observed at the indicated PS and, for those polymorphisms which result in amino acid variation, the identity of the alternative amino acids. In the screen exemplified in FIG. 7, the “Wild” column contains the number of individuals in the analyzed population homozygous for the wild-type, or the most common allele or reference allele. Similarly, the “Mut” column contains the number of individuals homozygous for the least common allele or uncommon variant allele, and the “Het” column contains the number of individuals heterozygous at that PS. The most and least common nucleotide (or encoded amino acid) at each site is defined by looking at the genotypes of all individuals in the population at that particular site. The nucleotide that shows up most often is called the most common nucleotide. The one that shows up less often is termed the least common. In situations where more than 2 nucleotides are seen at a site (which is rare but not unknown in human genes) all nucleotides except the most common one are lumped together in the least common category. The “Err” column indicates the number of individuals in which the variation in the “Change” column may have been incorrectly determined.

Checking a box in a row under the “Accept” column indicates that the haplotype is to include genotype information for the polymorphic site in that row. When a box under the “Accept” column is not checked, the genotype information concerning the polymorphic site described in that row will not be considered in the haplotype analysis for each of the individuals. For example, if a genotype has only one uncommon variant nucleotide (and, therefore, is not very informative for purposes of haplotype building), or if the genotype containing the polymorphism occurs in only one person, or does not obey Hardy-Weinberg equilibrium, it may be excluded from the analysis by not checking the relevant box for the polymorphism in the “Accept” column.

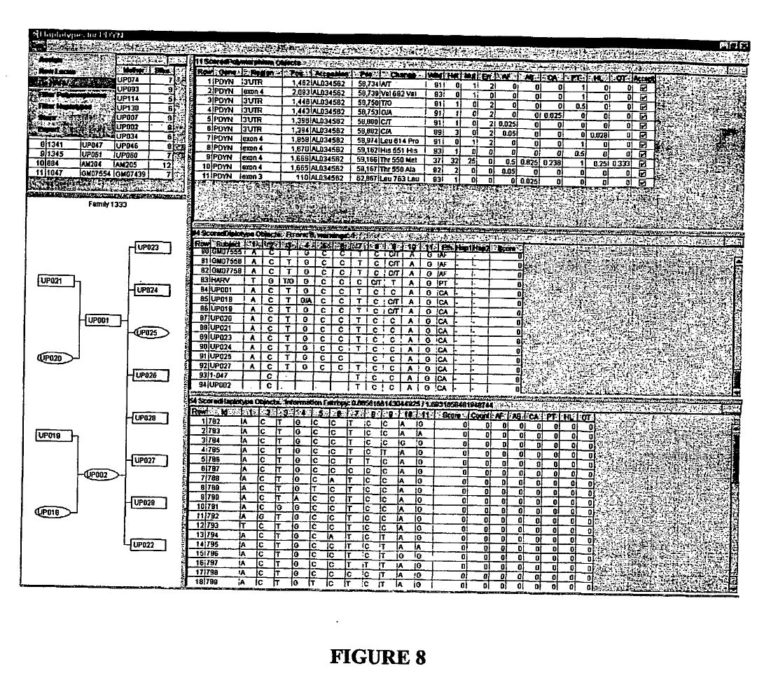

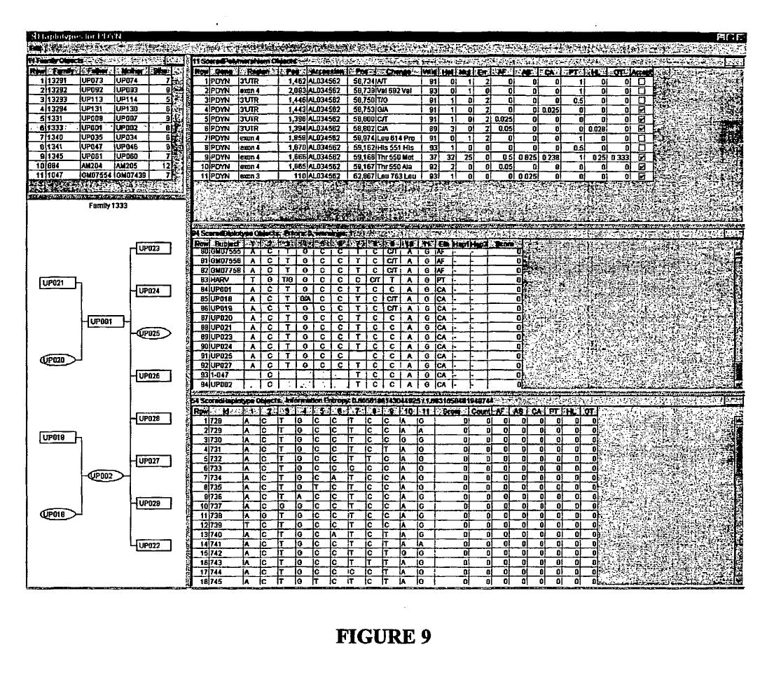

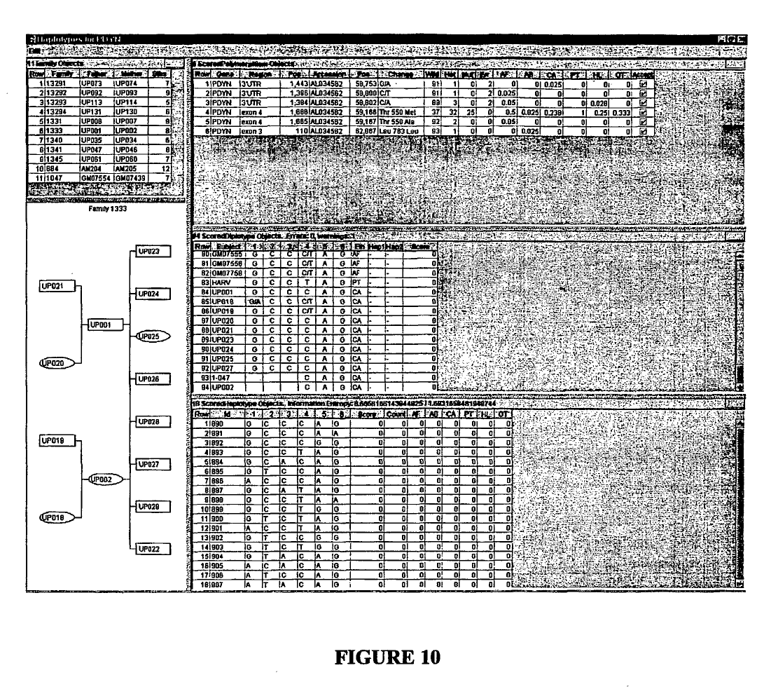

In addition, the screen exemplified in FIG. 7 displays the polymorphism frequency calculated for various groups of the analyzed population. In the screen, the different population groups are African American (AF), Asian (AS), Caucasian (CA), primate (PT; one chimpanzee individual named “Harv”) and other (OT; three native American individuals). The PDYN data set shown in FIG. 7 includes five “chimp-specific” polymorphic sites, i.e., the human individuals examined were all monomorphic at the position, but the chimpanzee had at least one alternative allele at that position. The rows containing these “chimp-specific” sites may be removed from this window by selecting the “Edit” button in the top left corner of the screen (which brings up the pull-down menu illustrated in FIG. 8), then selecting “De-HARV”, which unchecks the appropriate boxes in the “Accept” column (as shown in FIG. 9), and then selecting “Filter Polymorphisms”. The resulting human polymorphic sites for the PDYN data set are shown in FIG. 10. As indicated by comparing the “Scored Haplotype Objects” boxes of FIG. 9 and FIG. 10, the number of possible haplotypes expanded from the diplotypes goes down significantly (54 to 18), after De-HARV and filtering polymorphism steps were carried out for PDYN. In one embodiment, selecting “De-HARV” also hides the PT column. Alternatively, the program could be configured so that hiding the PT column and filtering of the “chimp-specific” sites would be accomplished in a one-step operation.

The box in the middle right side of the screen shown in FIG. 10 labeled “Scored Diplotype Objects” provides the genotype at each of the selected (accepted) polymorphic sites for each individual in the population being examined. For example, in this screen, the genotype data is shown for each of the 6 human polymorphic sites selected in the screen at the top of the figure for the PDYN in the indicated individuals from the Index Repository. Each row contains genotype information for a different individual and the genotypes for additional individuals in the population may be accessed by scrolling up and down, or by enlarging the window. The empty cells colored pink indicate those polymorphic sites for which sequence information is not present in the database. The “Subject” and “Eth” columns list the numerical identifier and ethnicity (i.e, population group) for the individual, respectively, using the same two-letter codes for the population groups described above. The “Hap1” and “Hap2” columns are empty in FIG. 10, but during the haplotype assignment process described above, these columns will indicate the most likely resolved haplotypes for the genotype for each individual in each row, based on the pair frequency score Fₖ determined for that pair by the method described herein, and listed in the “Score” column after each iteration of the haplotype assignment phase. As mentioned previously, this screen initially appears when the user clicks on the “Haplo” button in the screen shown in FIG. 7.

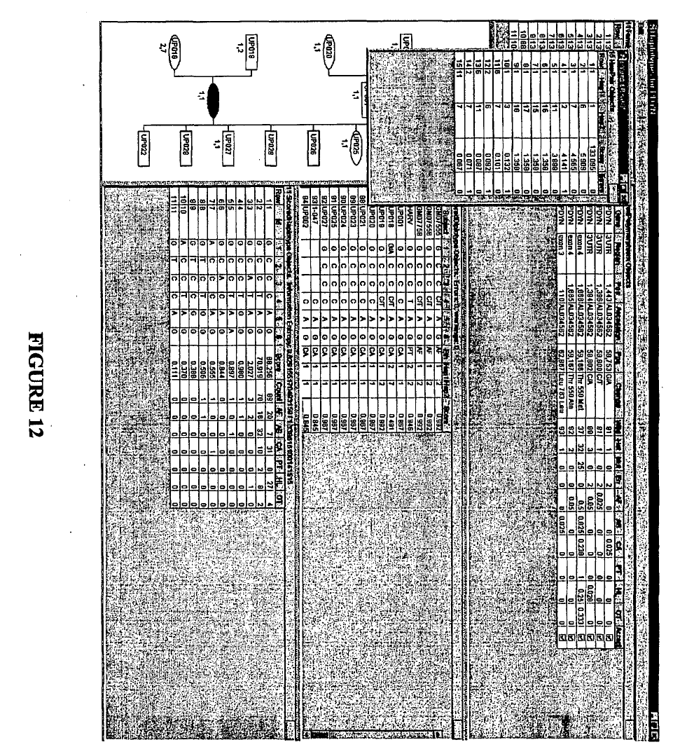

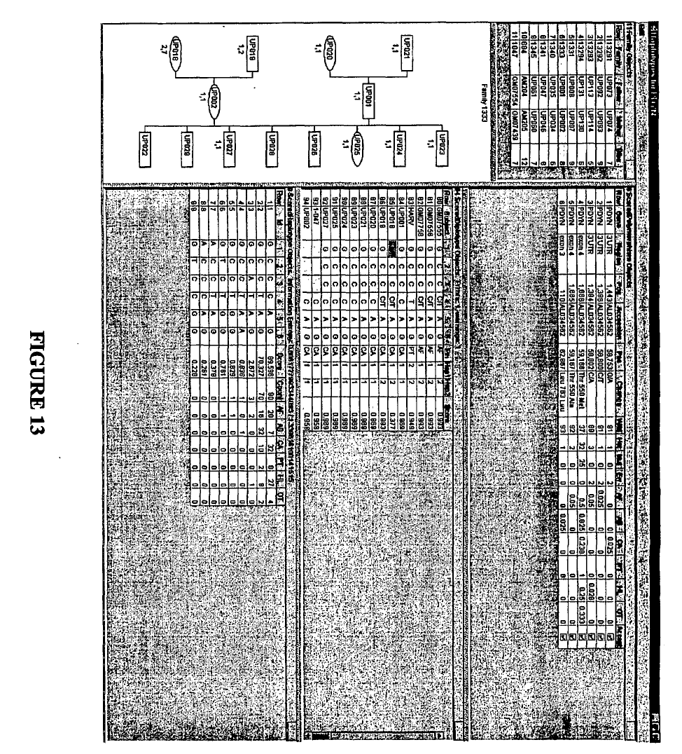

To begin the HAP™ Builder process, the user selects the “Assign” command in the pull-down menu in FIG. 10 (not shown). An example of a screen showing the result following one or more iterations of the haplotype pair assignment phase is shown, e.g., in FIGS. 12 and 13, respectively. The numbers in the “Hap1” and “Hap2” columns in the screens correspond to the HAP ID numbers in the window labeled “Scored Haplotype Objects” in the lower right side of the screens shown in FIGS. 12 and 13. For example, the genotype for individual UP018 in row 85 of the window in the middle right side of the screen in FIG. 12, the number 2 appears in the “Hap1” column and the number 7 appears in the “Hap2” column. This indicates that the initial most likely resolved haplotypes for individual UP018 are GCCTAG and ACCCAG, identified with ID numbers 2 and 7 respectively, in the window in the lower right had side of the screen. Compare the resolved haplotypes for individual UP018 in FIG. 13, after “Assign” has been selected a number of times. The number 1 appears in the “Hap 1” column and the number 2 appears in the “Hap 2” column. The score for this assignment is lower in FIG. 13 than in FIG. 12 and the genotype at polymorphic site 1 is highlighted in red as a possible “error” (e.g., a possible sequencing error).

The window labeled “Scored Haplotype Objects” (shown, e.g., in the lower right side of the screen exemplified in FIG. 13) provides the different haplotypes determined for the selected (accepted) polymorphic sites for the selected gene in the examined population. Each row contains a unique haplotype, with the current haplotype frequency score fᵢ of each haplotype listed in the “Score” column. The number of times each haplotype is seen in the entire population and in the various population groups are indicated in the “Count” and following six columns, respectively, with “AF”, “AS”, “CA”, “HL” and “OT” are as described above. The information in this window can also be sorted by haplotype frequency score fᵢ, by clicking on “Score”. In other embodiments, the PT and OT columns may be hidden manually or not considered in the HAP™ Builder process.

The “Information Entropy” shown at the top of the “ScoredHaplotypes Objects” window is a measure of the amount of variability of the locus. It measures the amount of information (in bits) that is needed to specify the genotype at the locus. If a locus has only one possible haplotype, there is only one possibility and the information entropy is zero. If there are four equally likely haplotypes, 2 bits of information are needed to specify which of the four is present. The general formula is

$${E}_{I} = \frac{1}{ln 2} \sum {k}_{i} ln {k}_{i} \qquad ( Formula 6 )$$

where kᵢ is the probability for a given possibility of outcome and the sum is over all possibilities. For a single polymorphism, there are only two possibilities, and the information entropy depends on the allele frequency k as

$${E}_{I} = \frac{1}{ln 2} ( k ln k + ( 1 - k ) ln ( 1 - k ) ) . \qquad ( Formula 7 )$$

If the polymorphism is balanced (k=0.5), E_{I} becomes one. If it is rare (k≅0), E_{I} approaches zero. The first number shown in the “ScoredHaplotpye objects” box is the information entropy of the locus as calculated from the possible haplotypes and their frequencies. The second number is the same quantity under the (erroneous) assumption that all polymorphisms are independent of each other. The former is always smaller than the latter and the difference indicates the degree to which the polymorphisms are linked. The largest possible information entropy is the number of polymorphisms N (if all N polymorphisms are balanced and independent of each other, or, in other words, if all 2ⁿ possible haplotypes are equally likely), more typically the values are between 0.5 and 3.

A large information entropy for a locus indicates greater variability, i.e., more haplotypes exist, and thus this locus may be more useful in finding associations with phenotypes than a locus with a smaller information entropy. This information is not used in building haplotypes.

Selecting the “Edit” menu in the top left corner of FIG. 8, for example, brings up the menu shown, having the following command selections: “Assign”; “New Locus”; “De-HARV”; “Filter Polymorphisms”; “Filter Haplotypes”; “Store”; and “Export”. Each invoking of the “Assign” command causes an additional iteration of the above-described haplotype assignment method to be carried out. Selecting “New Locus” clears out the scores and haplotype assignments and fills the “ScoredPolymorphism objects” box with data for all available polymorphisms for the locus. Selecting “De-HARV” removes the “Accept” checkmarks from those polymorphisms that are specific to the chimpanzee, i.e. those which are monomorphic in the human population. This selection is usually made when using the HAP™ Builder program, but does not need to be. The individual “Accept” checkmarks can also be modified manually. Selecting “Filter Polymorphisms” will eliminate all polymorphisms from the list and from the analysis which are not checked in the Accept column. Simultaneously, the Hap Expansion is performed and the resulting Haplotypes displayed in the “ScoredHaplotype objects” box. Selecting “Filter Haplotypes” allows the user to eliminate those haplotypes from the “ScoredHaplotype objects” box which have not been assigned as top choice to any individual. Selecting “Store” stores all the information into a database. This includes the list of haplotypes, the haplotype frequencies, and the haplotype pair assignments and assignment scores. Selecting “Export” allows the user to write the data into a text file, from which it can be read into a spreadsheet program or otherwise stored or transmitted.

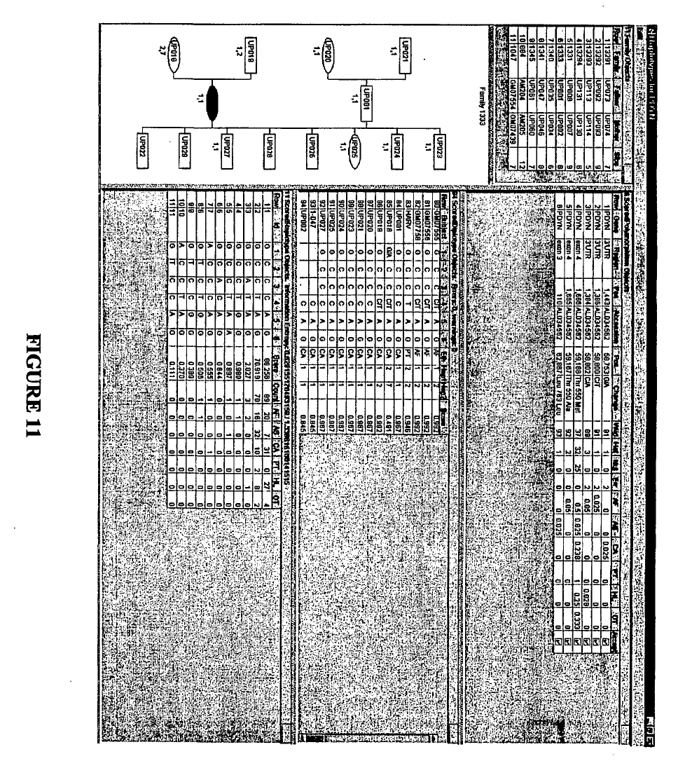

Clicking on the “Assign” command in the Edit Menu in FIG. 10 updates the boxes shown in the middle and lower boxes of FIG. 11. In the middle box on the right side of FIG. 9, Haplotypes have been assigned to each genotype (i.e., the “-”'s have been replaced by haplotype Id numbers in the “Hap 1” and “Hap 2” columns, and pair frequency scores have been assigned. In the bottom box on the right side of FIG. 11, haplotype frequency scores fᵢ have been assigned, as well as other information. There are 18 “Scored Haplotype Objects” shown in FIG. 10, but only 11 “Scored Haplotype Objects” shown in FIG. 11 because all haplotypes below the Hap frequency threshold of 0.1 have been dropped.

Clicking on 1333 in the Family Objects box in, e.g., FIGS. 11, 12 or 13 brings up the Family shown in the box on the top left side of those figures. The haplotypes assigned to the members of the family are indicated below the family members which were included in the HAP™ Builder process. FIG. 12 shows a screen on the bottom left labeled “HapPair Objects” which results when subject UP002 is selected in the screen in the “ScoredDiplotypes Objects” box in the middle right of FIG. 12. This contains the 15 most likely haplotype pairs for the individual UP002 based on the current haplotype pair scores Fₖ which are shown in the center right box. The pairs are shown with their pair probability scores in the “Score” column in the upper left box labeled “Hap Pair Objects”. The “Err” column indicates the number of positions at which the haplotype pair is not consistent with the measured genotype. If a pair in the list is clicked on, it will rise to the top of the list and the selected individual is assigned that pair with a probability score of 1.

FIG. 13 shows the changes that occur to the screens after reiterations of steps (e) through (g) of the Haplotype Assignment phase of the invention described above. This occurs when the “Assign” command in the Edit menu of FIG. 12 is invoked. Note the revised Scores in the boxes on the right side of the screen. A revised Score would also be visible on the HapPair Objects box (not shown).

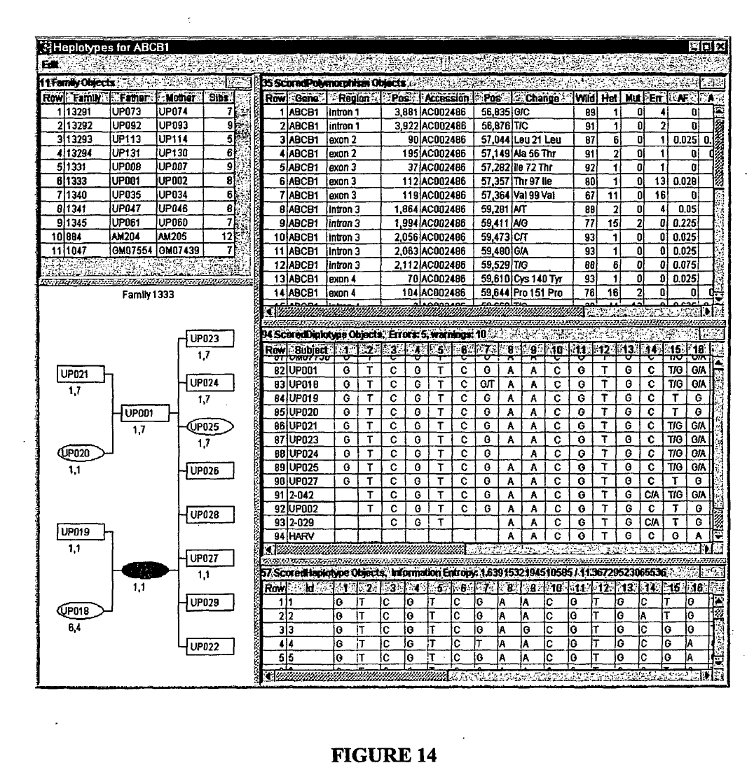

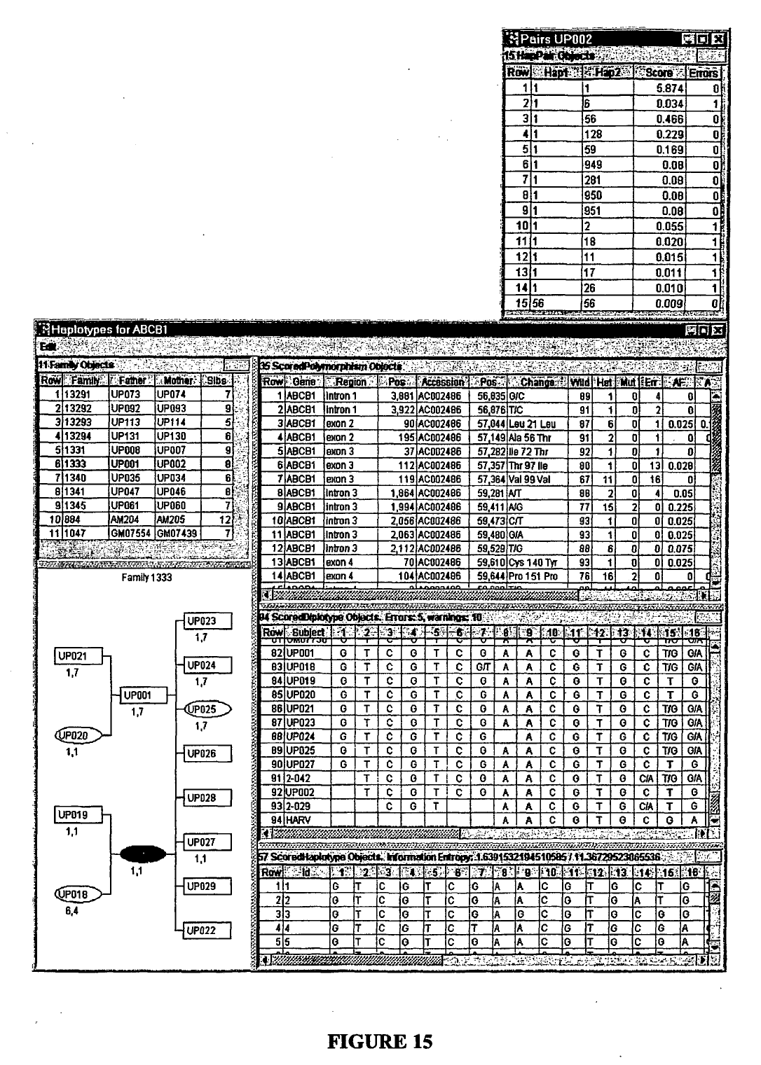

FIG. 13 shows the changes that occur to the screens after the iteration process is completed (following multiple selections of “Assign” and optional manual interventions), and the “Filter Haplotypes” options is selected. Only six Scored Haplotype Objects are shown in FIG. 13 as compared to eleven in FIG. 12, because all haplotypes not assigned to at least one individual have been dropped. Missing genotype data appears as blanks in the “Scored Diplotype Objects” box. FIGS. 14 and 15 show such blanks for the ABCB1 gene. The header of the center right box indicates that there are 10 warnings, flagged by boxes highlighted in pink in the ScoredDiplotype Objects box, and 5 errors, flagged by boxes highlighted in red. (Not all rows are visible in the Figures.)

FIG. 14 shows a situation where the assigned haplotypes do not obey Mendelian segregation in one of the families (Family 1333). The individual UP002, whose symbol has been flagged by red coloring, has been called as a 1,1 genotype (homozygous for haplotype 1). She could have inherited one haplotype 1 from her father, but could not have inherited the other from her mother, since her mother was called as a 4,6 genotype. The operator may conclude that the mother should be assigned a different pair such as 1,4 or 1,6; or may conclude that a different pair containing at least one copy of haplotype 1 needs to be assigned to the grandmother (UP018).

FIG. 15 shows how manual intervention can be used to fix the problem. The “HapPair Objects” window at the top of the figure has been brought up by clicking on UP002 (Row 92 in the “ScoredDiplotype Objects” box). By clicking on the second pair (Row 2) in this window, haplotype pair 1,6 can be assigned to subject UP002, and the requirements of Mendelian inheritance can be satisfied. The flag (red color) will then disappear from the family tree, but an additional error will appear in the “ScoredDiplotype Objects” window at the position which had to be overridden to accommodate the non-matching pair. In this particular case, it is more likely that the (6,4) haplotype assignment of the grandmother (UP018) is incorrect, since neither her daughter nor any of her grandchildren have either the 4 or the 6 haplotype. Manual intervention is useful to address such ambiguities.

The information that is stored in a database, such as a database associated with the DecoGen™ program exemplified herein includes (1) the positions of one or more, preferably two or more, most preferably all, of the sites in the gene locus (or other loci) that are variable (i.e. polymorphic) across members of the reference population and (2) the nucleotides found for each individuals' 2 haplotypes at each of the polymorphic sites. Preferably, it also includes individual identifiers and ethnicity or other phenotypic characteristics (such as age, gender or clinical information, if any) of each individual.

In the preferred embodiment of the invention, the haplotypes, their frequencies, and other information about each of the members of the population being analyzed, are stored and displayed, preferably in the manner shown, e.g. in FIGS. 7-15. The information shown in FIGS. 7-15 includes a unique identifier (shown in the “Subject” column), ethnicity, genotype, and (in FIGS. 11-15) the 2 haplotypes predicted for each individual. Only some of the individuals are visible in the screen. Scrolling up or down with the scroll bar brings information for other individuals into view. The subjects seen in these figures are from a reference population of healthy individuals.

V. TOOLS OF THE INVENTION

The methods of the invention preferably use a tool called the DecoGen™ program described in U.S. application Ser. No. 60/141,521, filed Jun. 25, 1999, and international application WO 01/01218, which are incorporated herein by reference.

The tool consists, in part, of:

a. One or more databases that contain (1) genotypes (or haplotypes) for a gene (or other loci) for many individuals (i.e., people, animals, plants, etc., depending on the application) for one or more genes and, optionally, (2) a list of the names or functions of the genes (or other loci), whose functions can be, but are not limited to: disease causation, drug response, plant yields, plant disease resistance, plant drought resistance, plant interaction with pest-management strategies, etc. The databases could include information generated either internally or externally (e.g. GenBank). Examples of databases which may be used in the present invention are described in U.S. application Ser. No. 60/141,521, filed Jun. 25, 1999, and international application WO 01/01218, which are incorporated herein by reference.

b. A set of computer programs that analyze and display the relationships between the genotypes and the haplotypes for an individual.

The methods of the invention preferably also use a tool called the HAP™ Builder Program. Specific aspects of this tool which are novel include:

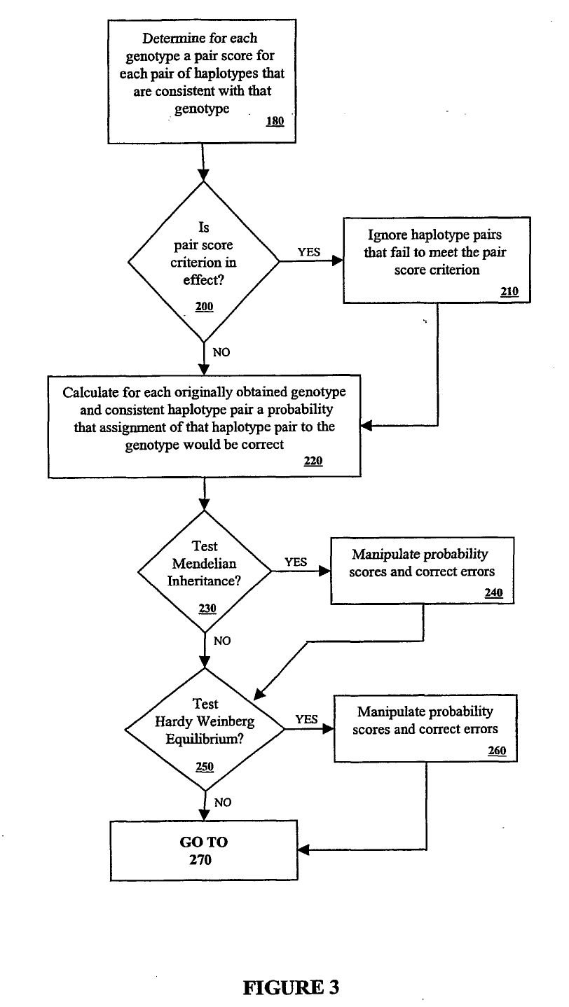

a. A new genotype-to-haplotype method that allows the user to infer an individual's haplotypes or sub-haplotypes for a given gene. The steps required for this to work are (a) determine the haplotype (or sub-haplotype) frequencies from the reference population by expanding the genotypes of a reference population; (b) optionally, correct the observed frequencies to conform to Hardy-Weinberg equilibrium and/or Mendelian inheritance (unless it is determined that the deviation is not due to sampling bias, sequencing error or questionable paternity); and (c) use the statistical approach described in this application (and shown schematically in FIGS. 2-4) to predict individuals' haplotypes or sub-haplotypes from their genotypes.

b. A method of displaying measurements of the probability of the correctness of the assignment of haplotypes or sub-haplotypes to individuals, as well as the ability to manually change the genotype, the haplotype pair assignments, and the probability of the assignments.

VI. DATA/DATABASE MODEL

The preferred embodiment present invention uses a relational database which provides a robust, scalable and releasable data storage and data management mechanism. The computing hardware and software platforms, with 7×24 teams of database administration and development support, provide the relational database with advantageous guaranteed data quality, data security, and data availability. The database model of the present invention provides tables and their relationships optimized for efficiently storing, searching and otherwise utilizing a genomics-oriented database.

A data model (or database model) describes the data fields one wishes to store and the relationships between those data fields. The model is a blueprint for the actual way that data is stored, but is generic enough that it is not restricted to a particular database implementation (e.g., Sybase™ or Oracle™). In the preferred embodiment of the present invention, the model covers the data required by, and/or generated by, the HAP™ Builder program. It contains at least 4 submodels which contain logically related subsets of the data. These relevant submodels, which are described in U.S. application Ser. No. 60/141.521, filed Jun. 25, 1999, and international application WO 01/01218, are described below.

1. Gene Repository: This is the sub-model that describes the gene loci and its related domains. Preferably, it captures the information on gene, gene structure, species, gene map, gene family, therapeutic applications of genes, gene naming conventions and published literature including the patent information on these objects.

2. Population Repository: This is the part of the data model that encapsulates the patient and population information. Preferably, it covers the entities such as patient, ethnic and geographical background of patient and population, medical conditions of the patients, family and pedigree information of the patients, patient haplotype and polymorphism information and their clinical trial outcomes.

3. Polymorphism Repository: This is the part of the model that covers the haplotype and the polymorphism associated with genes and, preferably, patient cohorts used in clinical studies. The polymorphisms include those due to single nucleotide polymorphisms (SNPs), large and small insertions and deletions, RFLPs, repeats, frame shifts and alternative splicings.

4. Sequence Repository: Genetic sequence information in the form of genomic DNA, cDNA, mRNA and protein is captured by this data model as is the location relationship between the gene structural features and the sequences.

VII. BUSINESS MODELS

The haplotype and other data developed using the methods and/or tools described herein may be used in a partnership of two or more companies (referred to herein as the Partnership) to integrate knowledge of human population and evolutionary variation into the discovery, development and delivery of pharmaceuticals, in the ways described in U.S. application Ser. No. 60/141,521, filed Jun. 25, 1999, and international application WO 01/01218, which are incorporated herein by reference.

The database and analytical tools of the invention are envisioned to be useful in a variety of settings, including various research settings, pharmaceutical companies, hospitals, independent or commercial establishments. It is expected users will include physicians (e.g., for diagnosing a particular disease or prescribing a particular drug) pharmaceutical companies, generics companies, diagnostics companies, contract research organizations and managed care groups, including HMOs, and even patients themselves.

However, as discussed above, it is obvious that various aspects of the invention may be useful in other settings, such as in the agricultural and veterinary venues.

The examples described herein illustrate certain embodiments of the present invention, but should not be construed as limiting its scope in any way. Certain modifications and variations will be apparent to those skilled in the art from the teachings of the foregoing disclosure and the following examples, and these are intended to be encompassed by the spirit and scope of the invention.

VIII. REFERENCES

1. Clark A G. (1990) Inference of haplotypes from PCR-amplified samples of diploid populations. Mol Biol Evol. 7:111-122.

2. Clark, A. G., et al. (1998) Haplotype structure and population genetic inferences from nucleotide-sequence variation in human lipoprotein lipase. Am. J. Hum. Genet., 63:595-612.

3. Dempster, A. P., et al. (1977). Maximum likelihood estimation from incomplete data via the EM algorithm. J. R. Stat. Soc. [B] 39:1-38.

4. Excoffier L, Slatkin M. (1995) Maximum-likelihood estimation of molecular haplotype frequencies in a diploid population. Mol Biol Evol 12:921-927.

5. Hawley M E, Kidd K K. (1995) HAPLO: a program using the EM algorithm to estimate the frequencies of multi-site haplotypes. J Hered 86:409-411.

6. Hill W G. (1975) Tests for association of gene frequencies at several loci in random mating diploid populations. Biometrics 31:881-888.

7. Long J C, Williams R C, Urbanek M. (1995) An E-M algorithm and testing strategy for multiple-locus haplotypes. Am J Hum Genet 56:799-810.

Modifications of the above described modes for carrying out the invention that are obvious to those of skill in the fields of chemistry, medicine, computer science and related fields are intended to be within the scope of the following claims.

What is claimed is:

1. A method for assigning haplotype pairs for a polymorphic genomic region to a plurality of individuals, comprising:
(a) obtaining a genotype for the polymorphic genomic region from each of the individuals;
(b) enumerating all possible haplotypes hᵢ that are consistent with each genotype;
(c) assigning an evidence score sᵢ to each of the enumerated haplotypes hᵢ;
(d) calculating an initial haplotype frequency fᵢ for each haplotype among the possible haplotypes, wherein the initial haplotype frequency fᵢ is a function of the evidence score sᵢ;
(e) determining for each genotype obtained in step (a) a pair score Fₖ for each pair of haplotypes that is consistent with that genotype, wherein Fₖ is a function of the frequency fᵢ for each of the haplotypes in the pair;
(f) calculating, for each genotype and consistent haplotype pair whose pair score Fₖ meets a pair score criterion, a probability pₖ that assignment of that haplotype pair to the genotype would be correct;
(g) generating a revised haplotype frequency fᵢ for each haplotype, wherein the revised haplotype frequency fᵢ is a function of the probability pₖ for each consistent haplotype pair which contains the haplotype; and
(h) repeating steps (e) through (g) until an end condition is reached, with the proviso that for each repetition the frequency fᵢ employed in step (e) is replaced by the revised frequency fᵢ determined in step (g).

2. The method of claim 1, wherein the evidence score sᵢ obeys a formula selected from the group consisting of:
0≦sᵢ≦2 and
$${\sum }_{i = 1}^{n} {s}_{i} = 2 ,$$
where n is the number of ambiguous positions in said genotype, wherein an ambiguous polymorphic site is either a heterozygous site or is a site for which nucleotide sequence information is lacking.

3. The method of claim 2 wherein the evidence score sᵢ is 2/2ⁿ, wherein n is the number of ambiguous positions in said genotype, with the proviso that if the polymorphic genomic region is haploid or hemizygous in the individual, an evidence score of 1 is assigned.

4. The method of claim 1 wherein the initial frequency fᵢ is calculated from the sum of the evidence scores across all the different individuals, for each of the possible haplotypes hᵢ.

5. The method of claim 1 wherein the enumerating step is applied only to each genotype that meets an ambiguity criterion.

6. The method of claim 5, wherein the ambiguity criterion is a function of the number of ambiguous polymorphic sites in the genotype, wherein an ambiguous polymorphic site is either a heterozygous site or is a site for which information is lacking.

7. The method of claim 1 wherein the pair score criterion is chosen from the group consisting of (a) a specific numerical cutoff; (b) a function of the values of the pair scores; and (c) a function of the rankings of the pair scores.

8. The method of claim 1, wherein the pair score Fₖ=2fᵢfⱼ, if i≢j, and otherwise Fₖ=fᵢ², with the proviso that the pair score Fₖ=fᵢ when the polymorphic genomic region is haploid or hemizygous, where fᵢ and fⱼ are the haplotype frequencies for the haplotypes hᵢ and hⱼ in the pair.

9. The method of claim 7, wherein the pair score criterion is a function of the rankings of the pair scores, and wherein the probability pₖ is calculated by:
(a) ranking each of the pair scores Fₖ by score, with the highest score first;
(b) disregarding all but the Nᵣₐₙₖ highest ranking assignments; and
(c) defining the probability pₖ as:
$${p}_{k} = \frac{{F}_{k}}{{\sum }_{l = 0}^{{N}_{rank}} {F}_{l}} ;$$

10. The method of claim 1, wherein the end condition is selected from the group consisting of: (i) steps (e) through (g) have been repeated a preset number of times; (ii) one or more of the parameters fᵢ, Fₖ, and pₖ has stabilized; and (iii) the operator choosses to stop.

11. The method of claim 1, wherein the plurality of individuals includes at least one multi-generation family and the probability pₖ is reduced for each pair assignment for each genotype in the family that does not obey Mendelian inheritance.

12. The method of claim 11, wherein the reduced probability is reduced to 0 or is reduced by the formula pₖ(1−pₖ′), where pₖ′ is the probability calculated for a pair assignment of a related genotype.

13. The method of claim 1, wherein the plurality of individuals comprises at least one population group and the probability pₖ is reduced for each pair assignment for each genotype in the population group that does not obey Hardy-Weinberg Equilibrium.

14. The method of claim 13, wherein the probability pₖ is reduced by the formula
$${p}_{k} ( 1 - \frac{{( {F}_{ii} - {f}_{i}^{2} )}^{2} + {( {F}_{ij} - 2 {f}_{i} {f}_{j} )}^{2} + {( {F}_{jj} - {f}_{j}^{2} )}^{2}}{{f}_{i}^{4} + 4 {f}_{i}^{2} {f}_{j}^{2} + {f}_{j}^{4}} ) ,$$
wherein fᵢ, and fⱼ are the frequencies of haplotypes hᵢ and hⱼ in the population group and Fᵢᵢ, Fⱼⱼ and Fᵢⱼ are the frequencies of each possible pair of haplotypes hᵢ and hⱼ in the population group.

15. The method of claim 1, wherein steps (a), (b) and (c) are performed for one individual at a time.

16. The method of claim 1, wherein steps (a), (b) and (c) are performed for each of the individuals in parallel.

17. The method of claim 1, wherein one or both of steps (e) and (f) are performed for one genotype at a time.

18. The method of claim 1, wherein one or both of steps (e) and (f) are performed for each genotype in parallel.

19. A method for predicting an individual's haplotype pair for a polymorphic genomic region, comprising:
(a) obtaining the genotype for the polymorphic genomic region from the individual;
(b) enumerating all possible haplotypes hᵢ for the genotype;
(c) providing a frequency fᵢ for each of the possible haplotypes, where fᵢ is determined by the method of claim 1,
(d) determining a pair score Fₖ for each pair of possible haplotypes hᵢ that are consistent with the genotype, wherein Fₖ is a function of the frequency fᵢ for each of the haplotypes in the pair; and
(e) assigning to the genotype the haplotype pair having the highest pair score Fₖ.

20. The method of claim 1, further including generating an error estimate.

21. A method of constructing a haplotype database for a population, comprising:
(a) determining haplotype data for a plurality of individuals from genotype information using the method of claim 1;
(c) organizing the haplotype data for the plurality of individuals into fields; and
(d) storing the haplotype data for the plurality of individuals according to the fields.

22. The method of claim 21, wherein the haplotype data comprises haplotype frequencies and haplotype pair scores for a polymorphic genomic region.

23. The method of claim 22, wherein the probabilities are reduced for haplotype pairs that do not meet the requirements of the Hardy-Weinberg equilibrium.

24. The method of claim 22 wherein the haplotype data further comprises probabilities that pair assignments are correct.

25. The method of claim 24, wherein the validating comprises correcting an observed distribution of haplotypes or haplotype pairs for effects imposed by a limited number of individuals in the population.

26. The method of claim 25, wherein the validating further comprises analyzing compliance of the observed distribution with Mendelian inheritance principles.

27. The method of claim 21, wherein the population is selected from the group consisting of a reference population, a clinical population, a disease population, an ethnic population, a family population and a same-sex population.

28. A method for predicting an individual's haplotype pair for a polymorphic genomic region, comprising
(a) identifying a genotype for the individual;
(b) enumerating all possible haplotype pairs which are consistent with the genotype;
(c) determining a probability for each possible haplotype pair that the individual has that possible haplotype pair by accessing a database containing frequency data for reference haplotype pairs; and
(d) analyzing the determined probabilities to predict an individual's haplotype pair.

29. The method of claim 28, further comprising storing the haplotype pair.

30. The method of claim 29, further comprising generating an error estimate.

31. A computer implemented method for generating haplotype pair and haplotype frequency screens for display on a display device, comprising the steps of:
(a) displaying in a first area a plurality of selectable items each corresponding to a polymorphic site for a predetermined gene;
(b) selecting one or more of said selectable items;
(c) displaying in a second area the haplotype pairs occurring in a reference population for the selected polymorphic sites;
(d) displaying in a third area data indicative of haplotype frequencies for a plurality of member groupings within the population.

32. A computer system for assigning haplotype pairs for a polymorphic genomic region to a plurality of individuals, comprising:
a database for storing genotyping information;
a processor connected to the database;
a computer program for controlling the processor connected to said database comprising instruction code to:
(a) accept input of a genotype for the polymorphic genomic region from each of the individuals and store said genotype within said database;
(b) enumerate all possible haplotypes hᵢ consistent with each genotype and store said haplotypes hᵢ within said database;
(c) calculate an evidence score sᵢ for each of said possible haplotypes hᵢ and store said evidence score sᵢ within said database;
(d) calculate an initial haplotype frequency fᵢ for each haplotype hᵢ among the possible haplotypes, and store the haplotype frequency fᵢ in said database, wherein the haplotype frequency fᵢ is a function of the evidence score sᵢ;
(e) calculate for each genotype received in step (a) a pair score Fₖ for each pair of haplotypes that are consistent with that genotype, wherein Fₖ is a function of the frequency fᵢ for each of the haplotypes in the pair;
(f) calculate, for each genotype and consistent haplotype pair whose pair score Fₖ meets a pair score criterion, a probability pₖ that assignment of that haplotype pair to the genotype would be correct and store the probability pₖ in said database;
(g) calculate a revised haplotype frequency fᵢ for each of the haplotypes, wherein the revised haplotype frequency fᵢ is a function of the probability pₖ for each consistent haplotype pair which contains the haplotype and storing the revised frequency fᵢ in said database; and
(h) repeat steps e through g until an end condition is reached, with the proviso that for each repetition the frequency fᵢ employed in step (e) is replaced by the revised frequency fᵢ determined in step (g) and stored in the database.

33. The computer system of claim 32 wherein the genotype for the polymorphic genomic region from each of the individuals is obtained electronically from a remote user and stored in said database.

34. The computer system of claim 33 wherein the computer system is connected to the internet and the genotype for the polymorphic genomic region from each of the individuals is obtained electronically from a remote user through the internet.

35. The computer system of claim 33 wherein the computer system is connected to the internet and the genotype for the polymorphic genomic region from each of the individuals is obtained electronically from a remote user through electronic mail.

36. The computer system of claim 33 wherein the genotype for the polymorphic genomic region from each of the individuals is obtained from a database of one or more known genotypes.

37. A computer readable medium comprising instruction code to:
(a) accept input of a genotype for the polymorphic genomic region from each of the individuals and store said genotype within said database;
(b) enumerate all possible haplotypes hᵢ consistent with each genotype and store said haplotypes hᵢ within said database;
(c) calculate an evidence score sᵢ for each of said possible haplotypes hᵢ and store said evidence score sᵢ within said database;
(d) calculate an initial haplotype frequency fᵢ for each haplotype hᵢ among the possible haplotypes, and store the haplotype frequency fᵢ in said database, wherein the haplotype frequency fᵢ is a function of the evidence score sᵢ;
(e) calculate for each genotype received in step (a) a pair score Fₖ for each pair of haplotypes that are consistent with that genotype, wherein Fₖ is a function of the frequency fᵢ for each of the haplotypes in the pair;
(f) calculate, for each genotype and consistent haplotype pair whose pair score Fₖ meets a pair score criterion, a probability pₖ that assignment of that haplotype pair to the genotype would be correct and store the probability pₖ in said database;
(g) calculate a revised haplotype frequency fᵢ for each of the haplotypes, wherein the revised haplotype frequency fᵢ is a function of the probability pₖ for each consistent haplotype pair which contains the haplotype and storing the revised frequency fᵢ in said database; and
(h) repeat steps e through g until an end condition is reached, with the proviso that for each repetition the frequency fᵢ employed in step (e) is replaced by the revised frequency fᵢ determined in step (g).

38. The method of any one of claims 1-27, wherein all of the individuals in the plurality of individuals meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.

39. The computer system of any one of claims 32-36, wherein all of the individuals in the plurality of individuals meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.

40. The computer-readable medium of claim 37, wherein all of the individuals in the plurality of individuals meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.

41. A method for assigning haplotype pairs for a polymorphic genomic region to a plurality of individuals, comprising:
(a) obtaining a genotype for the polymorphic genomic region from each of the individuals;
(b) grouping the genotypes obtained in step (a) into groups, wherein in each group g there are n_{g} identical genotypes, and wherein any unique genotypes are regarded as groups having n_{g}=1;
(c) enumerating all possible haplotypes hᵢ that are consistent with each distinct genotype;
(d) assigning an evidence score sᵢ to each of the enumerated possible haplotypes hᵢ;
(e) for each group g, calculating an initial haplotype frequency (fᵢ) for each haplotype among the possible haplotypes, wherein the initial haplotype frequency fᵢ is a function of the product (sᵢ)(n_{g});
(f) determining for each group g, a pair score Fₖ for each pair of haplotypes that is consistent with the genotype of that group, wherein Fₖ is a function of the frequency fᵢ for each of the haplotypes in the pair;
(g) calculating, for each genotype and consistent haplotype pair whose pair score Fₖ meets a pair score criterion, a probability pₖ that assignment of that haplotype pair to the genotype would be correct;
(h) generating a revised haplotype frequency fᵢ for each haplotype, wherein the revised haplotype frequency fᵢ is a function of the product (n_{g})(pₖ) for each consistent haplotype pair which contains the haplotype; and
(i) repeating steps (f) through (h) until an end condition is reached, with the proviso that for each repetition the frequency fᵢ employed in step (f) is replaced by the revised frequency fᵢ determined in step (h).

42. The method of claim 41, wherein the evidence score sᵢ obeys a formula selected from the group consisting of:
0≦sᵢ≦2 and
$${\sum }_{i = 1}^{n} {s}_{i} = 2 ,$$
where n is the number of ambiguous positions in said genotype, wherein an ambiguous polymorphic site is either a heterozygous site or is a site for which nucleotide sequence information is lacking.

43. The method of claim 42 wherein the evidence score sᵢ is 2/2ⁿ, wherein n is the number of ambiguous positions in said genotype, with the proviso that if the polymorphic genomic region is haploid or hemizygous in the individual, an evidence score of 1 is assigned.

44. The method of claim 41 wherein the initial frequency fᵢ is calculated from the sum of the products (n_{g})(sᵢ) across all the different genotypes, for all genotypes consistent with the haplotype fᵢ.

45. The method of claim 41 wherein the enumerating step is applied only to each genotype that meets an ambiguity criterion.

46. The method of claim 45, wherein the ambiguity criterion is a function of the number of ambiguous polymorphic sites in the genotype, wherein an ambiguous polymorphic site is either a heterozygous site or is a site for which information is lacking.

47. The method of claim 41 wherein the pair score criterion is chosen from the group consisting of (a) a specific numerical cutoff; (b) a function of the values of the pair scores; and (c) a function of the rankings of the pair scores.

48. The method of claim 41, wherein the pair score Fₖ=2fᵢfⱼ, if i≢j, and otherwise Fₖ=fᵢ², with the proviso that the pair score Fₖ=fᵢ when the polymorphic genomic region is haploid or hemizygous, where fᵢ and fⱼ are the haplotype frequencies for the haplotypes hᵢ and hⱼ in the pair.

49. The method of claim 47, wherein the pair score criterion is a function of the rankings of the pair scores, and wherein the probability pₖ is calculated by:
(a) ranking each of the pair scores Fₖ by score, with the highest score first;
(b) disregarding all but the Nᵣₐₙₖ highest ranking assignments; and
(c) defining the probability pₖ as:
$${p}_{k} = \frac{{F}_{k}}{{\sum }_{l = 0}^{{N}_{rank}} {F}_{l}} ;$$

50. The method of claim 41, wherein the end condition is selected from the group consisting of: (i) steps (e) through (g) have been repeated a preset number of times; (ii) one or more of the parameters fᵢ, Fₖ, and pₖ has stabilized; and (iii) the operator choosses to stop.

51. The method of claim 41, wherein the plurality of individuals includes at least one multi-generation family and the probability pₖ is reduced for each pair assignment for each genotype in the family that does not obey Mendelian inheritance.

52. The method of claim 51, wherein the reduced probability is reduced to 0 or is reduced by the formula pₖ(1−pₖ′), where pₖ′ is the probability calculated for a pair assignment of a related genotype.

53. The method of claim 41, wherein the plurality of individuals comprises at least one population group and the probability pₖ is reduced for each pair assignment for each genotype in the population group that does not obey Hardy-Weinberg Equilibrium.

54. The method of claim 53, wherein the probability pₖ is reduced by the formula
$${p}_{k} ( 1 - \frac{{( {F}_{ii} - {f}_{i}^{2} )}^{2} + {( {F}_{ij} - 2 {f}_{i} {f}_{j} )}^{2} + {( {F}_{jj} - {f}_{j}^{2} )}^{2}}{{f}_{i}^{4} + 4 {f}_{i}^{2} {f}_{j}^{2} + {f}_{j}^{4}} ) ,$$
wherein fᵢ, and fᵢ are the frequencies of haplotypes hᵢ and hⱼ in the population group and Fᵢᵢ, Fⱼⱼ and Fᵢⱼ are the frequencies of each possible pair of haplotypes hᵢ and hⱼ in the population group.

55. The method of claim 41, wherein steps (c), (d) and (e), are performed for one group at a time.

56. The method of claim 41, wherein steps (c), (d) and (e), are performed for each of the groups in parallel.

57. The method of claim 41, wherein one or both of steps (f) and (g) are performed for one genotype at a time.

58. The method of claim 42, wherein one or both of steps (f) and (g) are performed for each genotype in parallel.

59. A method for predicting an individual's haplotype pair for a polymorphic genomic region, comprising:
(a) obtaining the genotype for the polymorphic genomic region from the individual;
(b) enumerating all possible haplotypes hᵢ for the genotype;
(c) providing a frequency fᵢ for each of the possible haplotypes, where fᵢ is determined by the method of claim 41,
(d) determining a pair score Fₖ for each pair of possible haplotypes hᵢ that are consistent with the genotype, wherein Fₖ is a function of the frequency fᵢ for each of the haplotypes in the pair; and
(e) assigning to the genotype the haplotype pair having the highest pair score Fₖ.

60. The method of claim 41, further including generating an error estimate.

61. A method of constructing a haplotype database for a population, comprising:
(a) determining haplotype data for a plurality of individuals from genotype information using the method of claim 41;
(c) organizing the haplotype data for the plurality of individuals into fields; and
(d) storing the haplotype data for the plurality of individuals according to the fields.

62. The method of claim 61, wherein the haplotype data comprises haplotype frequencies and haplotype pair scores for a polymorphic genomic region.

63. The method of claim 61, wherein the probabilities are reduced for haplotype pairs that do not meet the requirements of the Hardy-Weinberg equilibrium.

64. The method of claim 61 wherein the haplotype data further comprises probabilities that pair assignments are correct.

65. The method of claim 61, wherein the validating comprises correcting an observed distribution of haplotypes or haplotype pairs for effects imposed by a limited number of individuals in the population.

66. The method of claim 65, wherein the validating further comprises analyzing compliance of the observed distribution with Mendelian inheritance principles.

67. The method of claim 61, wherein the population is selected from the group consisting of a reference population, a clinical population, a disease population, an ethnic population, a family population and a same-sex population.

68. A method for predicting an individual's haplotype pair for a polymorphic genomic region, comprising
(a) identifying a genotype for the individual;
(b) enumerating all possible haplotype pairs which are consistent with the genotype;
(c) determining a probability for each possible haplotype pair that the individual has that possible haplotype pair by accessing a database prepared by the method of claim 61 and containing frequency data for reference haplotype pairs; and
(d) analyzing the determined probabilities to predict an individual's haplotype pair.

69. The method of claim 68, further comprising storing the haplotype pair.

70. The method of claim 69, further comprising generating an error estimate.

71. A computer implemented method for generating haplotype pair and haplotype frequency screens for display on a display device, comprising the steps of:
(a) displaying in a first area a plurality of selectable items each corresponding to a polymorphic site for a predetermined gene;
(b) selecting one or more of said selectable items;
(c) displaying in a second area the haplotype pairs occurring in a reference population for the selected polymorphic sites;
(d) displaying in a third area data indicative of haplotype frequencies for a plurality of member groupings within the population;
wherein the data indicative of haplotype frequencies is retrieved from a database prepared by the method of claim 61.

72. A computer system for assigning haplotype pairs for a polymorphic genomic region to a plurality of individuals, comprising:
a database for storing genotyping information;
a processor connected to the database; and
a computer program for controlling the processor connected to said database, comprising instruction code to:
(a) accept input of a genotype for the polymorphic genomic region from each of the individuals and store said genotype within said database;
(b) group the genotypes input in step (a) into groups, wherein in each group g there are n_{g} identical genotypes, and wherein any unique genotypes are regarded as groups having n_{g}=1;
(c) enumerate all possible haplotypes hᵢ consistent with the genotype of each group g, and store said haplotypes hᵢ within said database;
(d) calculate an evidence score sᵢ for each of said possible haplotypes hᵢ and store said evidence score sᵢ within said database;
(e) for each group g, calculate an initial haplotype frequency fᵢ for each haplotype hᵢ among the possible haplotypes, and store the haplotype frequency fᵢ in said database, wherein the initial haplotype frequency fᵢ is a function of the product (sᵢ)(n_{g});
(f) calculate for each group obtained in step (b) a pair score Fₖ for each pair of haplotypes that are consistent with that group, wherein Fₖ is a function of the frequency fᵢ for each of the haplotypes in the pair;
(g) calculate, for each genotype and consistent haplotype pair whose pair score Fₖ meets a pair score criterion, a probability pₖ that assignment of that haplotype pair to the genotype would be correct and store the probability pₖ in said database;
(h) calculate a revised haplotype frequency fᵢ for each of the haplotypes, wherein the revised haplotype frequency fᵢ is a function of the product (n_{g})(pₖ) for each consistent haplotype pair which contains the haplotype and storing the revised frequency fᵢ in said database; and
(i) repeat steps (f) through (h) until an end condition is reached, with the proviso that for each repetition the frequency fᵢ employed in step (f) is replaced by the revised frequency fᵢ determined in step (h) and stored in the database.

73. The computer system of claim 72 wherein the genotype for the polymorphic genomic region from each of the individuals is obtained electronically from a remote user and stored in said database.

74. The computer system of claim 73 wherein the computer system is connected to the internet and the genotype for the polymorphic genomic region from each of the individuals is obtained electronically from a remote user through the internet.

75. The computer system of claim 73 wherein the computer system is connected to the internet and the genotype for the polymorphic genomic region from each of the individuals is obtained electronically from a remote user through electronic mail.

76. The computer system of claim 73 wherein the genotype for the polymorphic genomic region from each of the individuals is obtained from a database of one or more known genotypes.

77. A computer readable medium comprising instruction code to:
(a) accept input of a genotype for the polymorphic genomic region from each of the individuals and store said genotype within said database;
(b) group the genotypes input in step (a) into groups, wherein in each group g there are n_{g} identical genotypes, and wherein any unique genotypes are regarded as groups having n_{g}=1;
(c) enumerate all possible haplotypes hᵢ consistent with the genotype of each group g, and store said haplotypes hᵢ within said database;
(d) calculate an evidence score sᵢ for each of said possible haplotypes hᵢ and store said evidence score sᵢ within said database;
(e) for each group g, calculate an initial haplotype frequency fᵢ for each haplotype hᵢ among the possible haplotypes, and store the haplotype frequency fᵢ in said database, wherein the initial haplotype frequency fᵢ is a function of the product (sᵢ)(n_{g});
(f) calculate for each group obtained in step (b) a pair score pₖ for each pair of haplotypes that are consistent with that group, wherein Fₖ is a function of the frequency fᵢ for each of the haplotypes in the pair;
(g) calculate, for each genotype and consistent haplotype pair whose pair score Fₖ meets a pair score criterion, a probability pₖ that assignment of that haplotype pair to the genotype would be correct and store the probability pₖ in said database;
(h) calculate a revised haplotype frequency fᵢ for each of the haplotypes, wherein the revised haplotype frequency fᵢ is a function of the product (n_{g})(pₖ) for each consistent haplotype pair which contains the haplotype and storing the revised frequency fᵢ in said database; and
(i) repeat steps (f) through (h) until an end condition is reached, with the proviso that for each repetition the frequency fᵢ employed in step (f) is replaced by the revised frequency fᵢ determined in step (h) and stored in the database.

78. The method of any one of claims 41-71, wherein the groups are further characterized in that all the individuals, from whom the genotypes in the group are derived, meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.

79. The computer system of any one of claims 72-76, wherein the groups are further characterized in that all the individuals, from whom the genotypes in the group are derived, meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.

80. The computer-readable medium of claim 77, wherein the groups are further characterized in that all the individuals, from whom the genotypes in the group are derived, meet one or more criteria selected from the group consisting of:
(a) having the same gender;
(b) belonging to the same population group;
(c) belonging to the same clinical or disease population;
(d) exhibiting a particular response to a stimulus;
(e) having in common a particular genotype at a different polymorphic region; and
(f) having in common a particular haplotype at a different polymorphic region.
